(19) United States
(12) Patent Application Publication
(10) Pub. No.: US 2023/0383001 A1
(43) Pub. Date: Nov. 30, 2023

(54) METHOD FOR TREATING OX40 RELATED DISEASE

(71) Applicant: Kyowa Kirin Co., Ltd., Chiyoda-ku, Tokyo (JP)

(72) Inventors: Mitsuo SATO, Tokyo (JP); Yoshinori NAGATA, Tokyo (JP); Kenji OOTAKI, Tokyo (JP); Nobuyuki IMAI, Tokyo (JP); Munetake SHIMABE, Tokyo (JP)

(73) Assignee: Kyowa Kirin Co., Ltd., Chiyoda-ku, Tokyo (JP)

(21) Appl. No.: 18/248,238

(22) PCT Filed: Oct. 8, 2021
(86) PCT No.: PCT/JP2021/037481

§ 371 Date: Apr. 6, 2023

Related U.S. Application Data
(60) Provisional application No. 63/089,809, filed on Oct. 9, 2020.
(60) Provisional application No. 63/116,365, filed on Nov. 20, 2020.
(60) Provisional application No. 63/233,592, filed on Aug. 16, 2021.

(51) Int. Cl. C07K 16/28; A61P 17/00

(52) U.S. Cl. CPC C07K 16/2875; A61P 17/00; A61K 2039/505

(57) ABSTRACT

The present disclosure relates to an anti-OX40 antibody for use in treatment or prevention of OX-40 related diseases such as atopic dermatitis. In particular, the present disclosure provides an administration schedule that treats atopic dermatitis with an anti-OX40 antibody.

This application claims priority to U.S. provisional applications 63/089,809, filed Oct. 9, 2020, 63/116,365, filed Nov. 20, 2020, and 63/233,592, filed Aug. 16, 2021, the entire contents of which are incorporated herein by reference.

### TECHNICAL FIELD

The present disclosure relates to an anti-OX40 antibody for use in treatment or

prevention of OX40 related diseases such as atopic dermatitis. In particular, the present disclosure provides an administration schedule that treats atopic dermatitis with an anti-OX40 antibody.

### BACKGROUND

Atopic dermatitis (AD) is the most common chronic inflammatory skin disease, affecting both adults and children with worldwide prevalence rates of up to 20% (NPL 1). The standard care for skin inflammation includes topical treatments such as topical corticosteroids or tacrolimus ointment. While oral therapy including cyclosporine and systemic corticosteroids can be effective in cases of AD refractory to topical treatments, there is a need for improved medicines to treat AD refractory to topical treatments.

Activation of the T-cell subsets such as Th2 by OX40 (CD134) may play a role in the pathology of inflammatory skin diseases such as AD.

OX40 (CD134) is a member of the tumor necrosis factor (TNF) receptor gene family. OX40 is predominantly expressed early after antigen activation of T cells, including CD4 and CD8 positive T cells; T-helper type 1, type 2, and type 17 cells; and forkhead box P3 (FoxP3) positive/CD4 positive regulatory T cells. OX40 is involved in antigen-specific T cell expansion and survival. The ligand of OX40 (OX40L) is expressed mainly on activated antigen presenting cells and endothelial cells during inflammation. Ligation of OX40 by OX40L leads to enhanced T cell survival and proliferation and drives beneficial inflammatory processes as well as pathological autoimmune diseases.

Blocking the OX40/OX40L pathway has been shown to be protective against harmful T cell activation in several animal models of human disease such as asthma, inflammatory bowel disease, transplant rejection, autoimmune diabetes, graft versus host disease (GvHD), arthritis, experimental autoimmune encephalomyelitis.

Treatment of patients suffering of an OX40-mediated disease or prevention of such disease requires the development of administration strategies and dosages of OX40 blocking agents. PTL 1 discloses antibodies that specifically bind OX40, but does not disclose administration strategies and dosages for using such antibodies to treat or prevent an OX40-mediated disease. In one approach, PTL 2 provides a planned test of an administering anti-OX40 antibody over at most 16 weeks to treat atopic dermatitis. However, PTL 2 did not contemplate administering anti-OX40 antibody for a longer time period than 16 weeks, and did not report any results of administering the anti-OX40 antibody to human subjects.

Accordingly, there remain a need for an administration method for delivering anti-OX40 antibody for treating an OX40-mediated disease such as AD in a subject in need thereof that is proven to be safe and effective.

### CITATION LIST

### Patent Literature

PTL 1: US 2010/0196359

PTL 2: WO 2019/229155

### Non Patent Literature

NPL 1: Nakagawa et al. J. of Dermatological Science, 99: 82-89, 2020

### SUMMARY OF INVENTION

One embodiment of the present disclosure relates to a therapeutic method for an OX40-related immune- or allergy-related disease including subcutaneously administering an anti-OX40 antibody to a patient at a dose of 150 mg to 600 mg once in two weeks to four weeks for at least 16 weeks. In another embodiment, the present disclosure relates to a composition for use in the treatments of an OX40-related immune- or allergy-related disease, wherein an anti-OX40 antibody is subcutaneously administering to a patient at a dose of 150 mg to 600 mg once in two weeks to four weeks continuously at the same dose.

In some embodiments, the anti-OX40 antibody is a monoclonal antibody containing a heavy chain variable region (also called VH) containing the amino acid sequence of SEQ ID NO: 1 and a light chain variable region (also called VL) containing the amino acid sequence of SEQ ID NO: 2.

In some embodiments, the administration is continued for at least 20 weeks, 22 weeks, 24 weeks or 34 weeks after starting the administration.

In some embodiments, the OX40-related immune- or allergy-related disease is atopic dermatitis.

In some embodiments, the anti-OX40 antibody is subcutaneously administered once in two weeks, three weeks or four weeks.

In some embodiments, the dose is selected from 150 mg, 300 mg, 450 mg and 600 mg.

In some embodiments, the OX40-related immune- or allergy-related disease is moderate to severe atopic dermatitis.

In some embodiments, the OX40-related immune- or allergy-related disease is moderate to severe atopic dermatitis which is poorly controllable using a topical agent or moderate to severe atopic dermatitis for which a topical therapy is not medically recommended.

In some embodiments, the present disclosure relates to a therapeutic method or a composition for use in treating an OX40-related immune- or allergy-related disease, wherein the anti-OX40 antibody is combined with a known topical agent such as a steroid.

In some embodiments, the anti-OX40 antibody is KHK4083.

In some embodiments, the present disclosure relates to a therapeutic method for an OX40-related immune- or allergy-related disease including subcutaneously administering an anti-OX40 antibody to a patient at a dose of 150 mg to 600 mg once in two weeks to four weeks continuously at the same dose. In some embodiments, the present disclosure relates to a composition for use in a method for treating an OX40-related immune- or allergy-related disease including subcutaneously administering an anti-OX40 antibody to a patient at a dose of 150 mg to 600 mg once in two weeks to four weeks continuously at the same dose. In some embodiments, the anti-OX40 antibody is a monoclonal antibody containing a heavy chain variable region (also called VH) containing the amino acid sequence of SEQ ID NO: 1 and a light chain variable region (also called VL) containing the amino acid sequence of SEQ ID NO: 2. In some embodiments, the administration is continued for at least 16 weeks, 20 weeks, 22 weeks, 24 weeks or 34 weeks after starting the administration. In some embodiments, the immune- or allergy-related disease is atopic dermatitis. In some embodiments, the anti-OX40 antibody is subcutaneously administered once in two weeks, three weeks or four weeks. In some embodiments, the dose is selected from 150 mg, 300 mg, 450 mg and 600 mg. In some embodiments, the anti-OX40 antibody is KHK4083. In some embodiments, 300 mg of the anti-OX40 antibody is administered once in 2 weeks for 24 weeks, and then administered once in 4 weeks. In some embodiments, 300 mg of the anti-OX40 antibody is administered once in 2 weeks for 24 weeks, and then administered once in 8 weeks. In some embodiments, 300 mg of the anti-OX40 antibody is administered once in 2 weeks for 16 weeks and then administered once in 4 weeks. In some embodiments, 300 mg of the anti-OX40 antibody is administered once in 2 weeks for 16 weeks and then administered once in 8 weeks. In some embodiments, 150 mg of the anti-OX40 antibody is administered once in 2 weeks for 24 weeks, and then administered once in 4 weeks. In some embodiments, 150 mg of the anti-OX40 antibody is administered once in 2 weeks for 24 weeks, and then administered once in 8 weeks. In some embodiments, 150 mg of the anti-OX40 antibody is administered once in 2 weeks for 16 weeks and then administered once in 4 weeks. In some embodiments, 150 mg of the anti-OX40 antibody is administered once in 2 weeks for 16 weeks and then administered once in 8 weeks.

### BRIEF DESCRIPTION OF DRAWINGS

FIG. 1 shows a summary of the trial design.

FIG. 2 is a graph depicting proportions of Achieved EASI-75 of administration groups.

FIG. 3 is a graph depicting percentage changes from baseline in EASI Scores of administration groups.

FIG. 4 is a graph depicting percentage changes from baseline in blood helper T cell counts of administration groups.

FIG. 5 is a graph depicting proportion of achievement for EASI-75 (each week).

FIG. 6 is a graph depicting percentage changes (%) from baseline in EASI scores of administration groups.

FIG. 7 is a graph depicting time (weeks) to relapse without KHK4083 administration for patients achieving EASI-75 at W36.

FIG. 8 is a graph depicting the percentage changes from baseline in the total OX40-positive helper T cell counts (%) in blood.

FIG. 9 is a graph depicting the percentage changes from baseline in counts of unoccupied OX40-positive helper T cells (%) in blood.

FIG. 10 is a graph depicting the percentage changes from baseline counts of OX40-positive cells (%) in upper dermis.

FIG. 11 is a graph depicting the percentage changes from baseline in TARC value (%) in blood.

### DESCRIPTION OF EMBODIMENTS

The present invention relates to an anti-OX40 antagonist antibody for use in the treatment of subjects suffering of an OX40-mediated disease or disorder. Also provided by the present disclosure is a method for treating an OX40 mediated disease or disorder by administering to a subject a therapeutically effective amount of the disclosed anti-OX40 antagonist antibody.

In one aspect, the present disclosure relates to a therapeutic method for an OX40-related immune- or allergy-related disease including subcutaneously administering an anti-OX40 antibody to a patient at a dose of 150 mg to 600 mg once in two weeks to four weeks for at least 16 weeks.

### Definitions

Technical and scientific terms used herein have the meanings commonly understood by one of ordinary skill in the art to which the present invention pertains, unless otherwise defined. Materials, reagents and the like to which reference is made in the following description and examples are obtainable from commercial sources, unless otherwise noted.

As used herein, the singular forms “a,” “an,” and “the” designate both the singular and the plural, unless expressly stated to designate the singular only.

The term “about” means that the number comprehended is not limited to the exact number set forth herein, and is intended to refer to numbers substantially around the recited number while not departing from the scope of the invention. As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

As used herein, the term “subject” includes any human or nonhuman animal. The term “nonhuman animal” includes all vertebrates, e.g., mammals and non-mammals, such as nonhuman primates, sheep, dogs, cats, horses, cows, chickens, amphibians, reptiles, etc. Preferably the subject is human.

A “patient” for the purposes of the present invention includes both humans and other animals, preferably mammals and most preferably humans. Thus the antibodies of the present invention have both human therapy and veterinary applications. The term “treatment” or “treating” in the present invention is meant to include therapeutic treatment, as well as prophylactic, or suppressive measures for a disease or disorder. Thus, for example, successful administration of an antibody prior to onset of the disease results in treatment of the disease. As another example, successful administration of an antibody after clinical manifestation of the disease to combat the symptoms of the disease comprises treatment of the disease.

“Treatment” and “treating” also encompasses administration of an antibody after the appearance of the disease in order to eradicate the disease. Successful administration of an antibody after onset and after clinical symptoms have developed, with possible abatement of clinical symptoms and perhaps amelioration of the disease, comprises treatment of the disease. Those “in need of treatment” include mammals already having the disease or disorder, as well as those prone to having the disease or disorder, including those in which the disease or disorder is to be prevented.

### OX40 and Anti-OX40 Antibodies

The term “human OX40” as used herein includes variants, isoforms, and species homologs of human OX40. The use of “human OX40” herein encompasses all known or as yet undiscovered alleles and polymorphic forms of human OX40. The terms “human OX40”, “OX40” or “OX40 Receptor” are used herein equivalently and mean “human OX40” if not otherwise specifically indicated.

OX40L is a member of the TNF superfamily and is also known as gp34 or CD252. OX40L has also been designated CD252 (cluster of differentiation 252) and has the sequence database accession number P23510 (Swiss-Prot) or Q6FGS4 (Uniprot). OX40L is expressed on the surface of activated B cells, T cells, dendritic cells and endothelial cells.

The term “anti-OX40 antibodies” include antibodies or a fragment thereof that binds to OX40 e.g. OX40 in isolated form. The term “antibody or fragment thereof that binds to human OX40” includes antibodies or antigenic binding fragments thereof that bind to variants, isoforms, and species homologs of human OX40. The anti-OX-40 antibodies may bind OX40 with an affinity (KD) of 200 nM or less, preferably 100 nM or less, more preferably 50 nM or less, more preferably 20 nM or less, more preferably 10 nM or less, even more preferably 5 nM or less.

The term “antagonistic antibody” is used herein to include an antibody that is capable of inhibiting and/or neutralizing the biological signaling activity of OX40, for example by blocking binding or substantially reducing binding of OX40 to OX40 ligand and thus inhibiting or reducing the signaling pathway triggered by OX40 and/or inhibiting or reducing an OX40-mediated cell response like lymphocyte proliferation, cytokine expression, or lymphocyte survival.

The term “antibody” as referred to herein includes whole antibodies and any antigen binding fragments or single chains thereof. An “antibody” refers to a glycoprotein comprising at least two heavy (H) chains and two light (L) chains inter-connected by disulfide bonds, or an antigen binding fragment thereof. Each heavy chain is comprised of a heavy chain variable region (abbreviated herein as VH) and a heavy chain constant region. The heavy chain constant region is comprised of three domains, CHI, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR) with are hypervariable in sequence and/or involved in antigen recognition and/or usually form structurally defined loops, interspersed with regions that are more conserved, termed framework regions (FR or FW). Each VH and VL is composed of three CDRs and four FWs, arranged from amino-terminus to carboxy-terminus in the following order: FW1, CDR1, FW2, CDR2, FW3, CDR3, FW4. The amino acid sequences of FW1, FW2, FW3, and FW4 all together constitute the “non-CDR region” or “non-extended CDR region” of VH or VL as referred to herein.

Antibodies are grouped into classes, also referred to as isotypes, as determined genetically by the constant region. Fluman constant light chains are classified as kappa (CK) and lambda (CX) light chains. Fleavy chains are classified as mu (m), delta (d), gamma (y), alpha (a), or epsilon (e), and define the antibody's isotype as IgM, IgD, IgG, IgA, and IgE, respectively. Thus, “isotype” as used herein is meant any of the classes and/or subclasses of immunoglobulins defined by the chemical and antigenic characteristics of their constant regions. The known human immunoglobulin isotypes are IgG1 (IGHG1), lgG2 (IGHG2), lgG3 (IGHG3), lgG4 (IGHG4), IgA1 (IGHA1), lgA2 (IGHA2), IgM (IGHM), IgD (IGHD), and IgE (IGHE). The so-called human immunoglobulin pseudo-gamma IGHGP gene represents an additional human immunoglobulin heavy constant region gene which has been sequenced but does not encode a protein due to an altered switch region (Bensmana M et al., (1988) Nucleic Acids Res. 16(7): 3108). In spite of having an altered switch region, the human immunoglobulin pseudo-gamma IGHGP gene has open reading frames for all heavy constant domains (CHI-CH3) and hinge. All open reading frames for its heavy constant domains encode protein domains which align well with all human immunoglobulin constant domains with the predicted structural features. This additional pseudo-gamma isotype is referred herein as IgGP or IGHGP. Other pseudo immunoglobulin genes have been reported such as the human immunoglobulin heavy constant domain epsilon PI and P2 pseudo genes (IGHEP1 and IGH EP2). The IgG class is the most commonly used for therapeutic purposes. In humans this class comprises subclasses IgG1, lgG2, lgG3 and lgG4. In mice this class comprises subclasses IgG1, lgG2a, lgG2b, lgG2c and lgG3.

The antibodies of the present disclosure may be an anti-OX40 antagonist antibody for use in the treatment of patients suffering of an OX40-mediated disorders. Also provided by the present disclosure is a method for treating an OX40 mediated disorder by administering to a patient a therapeutically effective amount of the disclosed anti-OX40 antagonist antibody.

In one aspect, the therapeutic method according to the present disclosure, an anti-OX40 antibody is used for treating an OX40-mediated disorder, wherein the anti-OX40 antibody is a monoclonal antibody containing a heavy chain variable region (also called VH) containing the amino acid sequence of SEQ ID NO: 1 and a light chain variable region (also called VL) containing the amino acid sequence of SEQ ID NO: 2.

Examples of the constant regions contained in the anti-OX40 antibody of the invention include a constant region containing the amino acid sequence of SEQ ID NO: 3 and a constant region containing the amino acid sequence of SEQ ID NO: 4. An example of the anti-OX40 antibody of the invention is a monoclonal antibody containing a heavy chain containing the amino acid sequence of SEQ ID NO: 5 and a light chain containing the amino acid sequence of SEQ ID NO: 6.

In some embodiments, the anti-OX40 antibody is KHK4083.

The complementarity determining region (CDR) sequences of the anti-OX40 antibody of the invention can be determined referring to the human framework (referred to as FR below) consensus sequences and the human antibody germline sequences reported by Kabat et al. [Sequences of Proteins of Immunological Interest, US Dept. Health and Human Services (1991)] as the amino acid sequences of human antibody FRs. In addition, the CDRs can also be defined by the ImMunoGeneTics (IMGT) numbering system. CDR sequences defined by Kabat numbering, IMGT numbering or any other known method using a heavy chain variable region (VH) containing the amino acid sequence of SEQ ID NO: 1 and a light chain variable region (VL) of SEQ ID NO: 2 are also included in the CDR sequences of the anti-OX40 antibody of the invention.

### OX40-Related Disease or Disorder

As used herein, the term “OX40-related disease” maybe any disease or disorder associated with aberrant OX40 signaling. The terms “OX40-related disease” and “OX40-mediated disease” are used interchangeable and are meant to be equivalent terms.

In some embodiments, the OX40-related disease may be a disease caused by harmful T cell activation mediated by OX40. In some embodiments, the OX40-related disease may be asthma, inflammatory bowel disease, transplant rejection, autoimmune diabetes, graft versus host disease (GvHD), arthritis, or experimental autoimmune encephalomyelitis.

In some embodiments, the therapeutic method disclosed herein may be used for treatment of OX40-related immune- or allergy-related disease, wherein the OX40-related immune- or allergy-related disease is atopic dermatitis. In some embodiments, the OX40-related immune- or allergy-related disease is moderate to severe atopic dermatitis which is poorly controllable using a topical agent or moderate to severe atopic dermatitis for which a topical therapy is not medically recommended. In some embodiments, the OX40-related immune- or allergy-related disease is moderate to severe atopic dermatitis.

Atopic dermatitis (AD), as used herein, means an inflammatory skin disease characterized by intense pruritus (e.g., severe itch) and by scaly and dry eczematous lesions. The term “atopic dermatitis” includes, but is not limited to, AD caused by or associated with epidermal barrier dysfunction, allergy (e.g., allergy to certain foods, pollen, mold, dust mite, animals, etc.), radiation exposure, and/or asthma. The present disclosure encompasses methods to treat patients with mild, moderate -to-severe or severe AD. As used herein, “moderate-to-severe AD”, is characterized by intensely pruritic, widespread skin lesions that are often complicated by persistent bacterial, viral or fungal infections. Moderate -to-severe AD also includes chronic AD in patients. In many cases, the chronic lesions include thickened plaques of skin, lichenification and fibrous papules. Patients affected by moderate-to-severe AD also, in general, have more than 10% of the body's skin affected, or 10% of skin area in addition to involvement of the eyes, hands and body folds.

The efficacy in the invention can be assessed based on an index, for example, EASI (Eczema area and severity index), SCORAD (Severity scoring of atopic dermatitis), IGA (Investigator's global assessment), BSA (Body Surface area), pruritus NRS (Numerical rating scale), sleep disturbance NRS, DLQI (Dermatology Life Quality Index), TARC (Thymus and activation-regulated chemokine), or the like, but the index is not limited thereto. An “improvement in an AD- related efficacy parameters ” means a decrease from baseline of one or more of IGA, BSA, EASI, SCORAD, TEWL, DLQI or pruritus NRS.

According to the treatment method of the present invention, the patient's EASI score can be reduced by at least 20% or more, 30% or more, 40% or more, 50% or more, 60% or more, 70% or more, 75% or more, or 80% or more from baseline.

Investigators Global Assessment (IGA): the IGA is an assessment scale used in clinical studies to determine severity of AD and clinical response to treatment based on a 5 -point scale ranging from 0 (clear) to 4 (severe/very severe). Each IGA scale is, for example, defined as follows:
0=Clear: No inflammatory signs of atopic dermatitis (no erythema, no induration/

papulation, no lichenification, no oozing/crusting). Postinflammatory hyperpigmentation and/or hypopigmentation may be present.
1=Almost clear: Barely perceptible erythema, barely perceptible induration/

papulation, and/or minimal lichenification. No oozing or crusting.
2=Mild: Slight but definite erythema (pink), slight but definite induration/

papulation, and/or slight but definite lichenification. No oozing or crusting.
3=Moderate: Clearly perceptible erythema (dull red), clearly perceptible induration/papulation, and/or clearly perceptible lichenification. Oozing and crusting may be present.
4=Severe: Marked erythema (deep or bright red), marked induration/papulation, and/or marked lichenification. Disease is widespread in extent. Oozing or crusting may be present.

The Eczema Area and Severity Index (EASI) is a validated measure used in clinical practice and clinical studies to assess the severity and extent of AD. Four AD disease characteristics will be assessed for severity by the investigator or designee on a scale of “0” (absent) through “3” (severe). In addition, the area of AD involvement will be assessed as a percentage by body area of head/neck, trunk (including genital area), upper limbs, and lower limbs (including buttocks), and converted to a score of 0 to 6 (Hanifin et al, 2001).

The SCORing Atopic Dermatitis Assessment (SCORAD) is a validated tool used in clinical research and clinical practice that was developed to standardize the evaluation of the extent and intensity of AD. The extent of AD is assessed as a percentage of each defined body area and reported as the sum of all areas, with a maximum score of 100% (assigned as “A” in the overall SCORAD calculation). The intensity of 6 specific symptoms of AD is assessed using the following scale: absence (0), mild (1), moderate (2), or severe (3), (for a maximum of 18 total points, assigned as “B” in the overall SCORAD calculation). Subjective assessment of pruritus and sleeplessness is recorded for each symptom by the subject or relative on a visual analogue scale (VAS), where 0 is no pruritus (or sleeploss) and 10 is the worst imaginable pruritus (or sleeploss), with a maximum possible score of 20. This parameter is assigned as “C” in the overall SCORAD calculation. The SCORAD is calculated as: A/5+7B/2+C (Kunz et al, Dermatology, 195(1):10-9 1997).

For the Pruritus Numerical Rating Scale (Pruritus NRS), subjects will respond to the question of their worst degree of itch during the previous 24 hours by choosing one of the scores from 0-10 with 0 being no itch and 10 being the worst itch imaginable.

The Dermatology Life Quality Index (DLQI) is a subject-administered, 10-question, validated, quality-of-life questionnaire that covers 6 domains including symptoms and feelings, daily activities, leisure, work and school, personal relationships, and treatment. Response categories include “a little,” “a lot,” and “very much” with corresponding scores of 1, 2, and 3, respectively; “not at all”, “not relevant” responses are scored as “0.” Totals range from 0 to 30 (ie, from less to more impairment) and a change from baseline is considered clinically relevant (Finlay and Khan, Clin Exp Dermatol., May; 19(3):210-6, 1994; Basra et al, Br J Dermatol., November; 159(5):997-1035).

For the Global Individual Signs Score (GISS), individual components of the AD lesions (erythema, infiltration/papulation, excoriations, and lichenification) will be rated globally (ie, each assessed for the whole body, not by anatomical region) on a 4-point scale (from 0=none to 3=severe) using the EASI severity grading criteria.

Body surface area (BSA) affected by AD will be assessed for each section of the body (the possible highest score for each region is: head and neck [9%], anterior trunk [18%], back [18%], upper limbs [18%], lower limbs [36%], and genitals [1%]) and will be reported as a percentage of all major body sections combined.

The Hospital Anxiety Depression Scale (HADS) is an instrument for screening anxiety and depression in non-psychiatric populations; repeated administration also provides information about changes to a patient's emotional state (Zigmond and Snaith, 1983; Herrmann, 1997). The HADS consists of 14 items, 7 each for anxiety and depression symptoms; possible scores range from 0 to 21 for each subscale. The following cut-off scores are recommended for both subscales: 7 to 8 for possible presence, 10 to 11 for probable presence, and 14 to 15 for severe anxiety or depression.

The Patient-Oriented Eczema Measure (POEM) is a 7-item, validated questionnaire used in clinical practice and clinical trials to assess disease symptoms in children and adults (Charman et al, 2004). The format is a response to 7 items (dryness, itching, flaking, cracking, sleep loss, bleeding, and weeping) based on frequency during the past week (i.e., 0=no days, 1=1 to 2 days, 2=3 to 4 days, 3=5 to 6 days, and 4=all days) with a scoring system of 0 to 28; the total score reflects disease-related morbidity.

The EuroQol-5D (EQ-5D) is a standardized measure of health status developed by the EuroQOL Group in order to provide a simple, generic measure of health for clinical and economic appraisal. The EQ-5D consists of 2 parts: the descriptive system and the EQ visual analogue scale (EQVAS). The EQ-5D descriptive system comprises the following 5 dimensions: mobility, self-care, usual activities, pain/discomfort, and anxiety/depression. Each dimension has 3 levels of perceived problems: “no problems” (level 1), “some problems” (level 2), “extreme problems” (level 3). The VAS scale is a 100-point scale with endpoints ranging from 100—“best imaginable health state” to 0—“worst imaginable health state”.

The Asthma Control Questionnaire-5 (ACQ-5) is a 5-question version of the Juniper ACQ is a validated questionnaire to evaluate asthma control. The questionnaire will be administered only to the subset of subjects with a medical history of asthma.

The Sino-nasal Outcome Test (SNOT-22) is a validated questionnaire to assess the impact of chronic rhinosinusitis on quality of life (QOL). The questionnaire will be administered only to the subset of subjects with chronic inflammatory conditions of the nasal mucosa and/or paranasal sinuses (eg, chronic rhinitis/rhinosinusitis, nasal polyps, allergic rhinitis).

Patient Global Assessment of Disease: subjects will rate their overall wellbeing based on a 5-point Likert scale from poor to excellent. Subjects will be asked: “Considering all the ways in which your eczema affects you, indicate how well you are doing.” Response choices are: “Poor”; “Fair”; “Good”; “Very Good”; “Excellent.”

Patient Global Assessment of Treatment: subjects will rate their satisfaction with the study treatment based on a 5-point Likert scale from poor to excellent. Subjects will be asked: “How would you rate the way your eczema responded to the study medication?” Response choices are: “Poor”; “Fair”; “Good”; “Very Good”; “Excellent”.

Atopic dermatitis biomarker parameters. The present invention also includes methods involving the use, quantification, and analysis of Atopic dermatitis biomarker parameters. As used herein, the term “Atopic dermatitis biomarker parameters ” means any biological response, cell type, parameter, protein, polypeptide, enzyme, enzyme activity, metabolite, nucleic acid, carbohydrate, or other biomolecule which is present or detectable in an AD patient at a level or amount that is different from (e.g., greater than or less than) the level or amount of the marker present or detectable in a non-AD patient. In some embodiments, the term “Atopic dermatitis biomarker parameters” includes a biomarker associated with Type 2 helper T-cell Th2)-driven inflammation. In order to evaluate for the drug effect or how much of the disease profile has been reversed by treatment as measured changes in the AD transcriptome using gene arrays consisting of differentially expressed genes between lesional and non lesional AD skin as defined by fold changes (typically a fold change of more than 2). The AD disease phenotype is the integration of cellular and molecular markers that define the epidermal pathology (hyperplasia, differentiation abnormalities), and Th2, and Th22 immune activation. The changes or reversal of these immune and barrier defects will be assessed by IHC and RT-PCR.

Other exemplary AD-associated biomarkers include a panel of Th1, Th2, Th22, Th17/Th22 cytokines and chemokines e.g., K16, Ki67, IFNy, CXCL10, IL-31, IL-4, IL-13, CCL11, CCL17, TSLP, IL-23p19, IL-8, and S100As, Serum Thymus and activation-regulated chemokine (TARC/CCL17), eotaxin-3, total Immunoglobulin E (IgE), Thymus and activation-regulated chemokine is a chemokine, shown to be strongly associated with disease severity in AD, and may be involved in pathogenesis of the disease.

Baseline TARC levels will be assessed for potential predictive value for treatment response. Eotaxin-3 (CCL26), Eotaxin-3 is a chemokine, shown to be associated with disease severity in AD, and may be involved in pathogenesis of the disease. Baseline eotaxin-3 levels will be assessed for potential predictive value for treatment response. Post-treatment samples will be evaluated for effects of anti OX40 antagonist antibody on eotaxin-3. Total Immunoglobulin E (IgE), Patients with AD often have elevated IgE. Total IgE levels have been found to modestly correlate with AD severity and may be involved in the pathogenesis of the disease. Changes in total IgE reflects not only on AD, but atopy in general. Baseline IgE levels will be assessed for potential predictive value for treatment response. Trans -epidermal water loss (TEWL). Transepidermal water loss is a skin barrier function test that measures perspiration or water loss through the skin. This procedure involves the non-invasive application of a probe on the surface of the skin on the arm or leg. Affected and non-affected areas of skin will be tested.

In some embodiments, the OX40-related immune- or allergy-related disease is moderate to severe atopic dermatitis which is poorly controllable using a topical agent or moderate to severe atopic dermatitis for which a topical therapy is not medically recommended. In other embodiments, the OX40-mediate disease comprises infections (viral, bacterial, fungal and parasitic, endotoxic shock associated with infection, arthritis, rheumatoid arthritis, asthma, bronchitis, influenza, respiratory syncytial virus, pneumonia, COPD, idiopathic pulmonary fibrosis (IPF), cryptogenic fibrosing alveolitis (CFA), idiopathic fibrosing interstitial pneumonia, emphysema, pelvic inflammatory disease, Alzheimer's Disease, inflammatory bowel disease, Crohn's disease, ulcerative colitis, Peyronie's Disease, coehac disease, gallbladder disease, Pilonidal disease, peritonitis, psoriasis, vasculitis, surgical adhesions, stroke, Type I Diabetes, lyme disease, arthritis, meningoencephalitis, autoimmune uveitis, immune mediated inflammatory disorders of the central and peripheral nervous system such as multiple sclerosis, lupus (such as systemic lupus erythematosus) and Guillain-Barr syndrome, Atopic dermatitis, wherein atopic dermatitis is mild, or mild-to-moderate, or moderate, or moderate-to-severe, or severe, autoimmune hepatitis, fibrosing alveolitis, Grave's disease, IgA nephropathy, idiopathic thrombocytopenic purpura, Meniere's disease, pemphigus, primary biliary cirrhosis, sarcoidosis, scleroderma, Wegener's granulomatosis, other autoimmune disorders, pancreatitis, trauma (surgery), graft-versus-host disease (GVHD), transplant rejection, cardiovascular disease including ischaemic diseases such as myocardial infarction as well as atherosclerosis, intravascular coagulation, bone resorption, osteoporosis, osteoarthritis, periodontitis, hypochlorhydia, hidradenitis and neuromyelitis optica.

### Administration and Dosage Regimen

The antibody or composition of the present invention can be administered via one or more routes of administration using one or more of a variety of methods known in the art. As will be appreciated by the skilled artisan, the route and/or mode of administration will vary depending upon the desired results. Preferred routes of administration include intravenous, intramuscular, intradermal, intraperitoneal, subcutaneous, spinal or other parenteral routes of administration, for example by injection or infusion. More preferred routes of administration are intravenous or subcutaneous. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, sub-cuticular, intraarticular, subcapsular, subarachnoid, intraspinal, epidural and intrasternal injection and infusion. Alternatively, an antibody of the invention can be administered via a non-parenteral route, such as a topical, epidermal or mucosal route of administration, for example, intranasally, orally, vaginally, rectally, sublingually or topically. In a preferred aspect of the present invention the anti-OX40 antagonist antibody is administered subcutaneously.

The administration of drugs, including the anti-OX40 antagonist antibody, may be described in terms of pharmacokinetics (PK) parameters. Non limiting examples of PK parameters include: maximum observed serum concentration (Cmax), average plasma drug concentration (Cavg), trough plasma concentration (Ctrough), last measurable plasma concentration (Clast), area under the plasma concentration-time curve at time t (AUCt), e.g. AUC168 being the area under the concentration-time curve (time 0 to time 168 hours)area under the serum concentration time curve from time 0 to time of the last measurable concentration (AUCO-last), area under the plasma concentration-time curve from time zero to infinity (AUCO-inf) time of maximum observed serum concentration (Tmax), time of last observed serum concentration (Tlast), apparent terminal elimination half-life (t1/2), total clearance (CL), apparent volume of distribution associated with the terminal phase (Vz), volume of distribution at steady state (Vss), accumulation ratio (Rac).

In particular, provided by the present disclosure is an anti-OX40 antagonist antibody for use in the treatment of an OX40-mediated disorder, wherein said anti-OX40 antibody is administered to a patient in need thereof intravenously or subcutaneously. Also provided by the present invention is a method for treating an OX40 mediated disorder, wherein said anti-OX40 antibody is administered to a patient in need thereof intravenously or subcutaneously.

The antibody or composition of the present disclosure can be administered at a single or multiple doses. The term “dose” as used in the present invention, indicates an amount of drug substance administered per body weight of a subject or a total dose administered to a subject irrespective to their body weight.

In an embodiment , the dose is a dose selected from 50 to 1000 mg but is preferably a dose selected from 75 to 600 mg, more preferably a dose selected from 100 to 600 mg, further preferably a dose selected from 150 to 600 mg.

In another embodiment of the invention, the dose may be any of 50, 75, 100, 150, 200, 250, 300, 350, 400, 450, 500, 550 and 600 mg but is preferably 150, 300, 450 or 600 mg, more preferably 150, 300 or 600 mg.

In an embodiment of the invention, after subcutaneously administering the anti-OX40 antibody for at least 16 weeks, the anti-OX40 antibody may be continuously administered but does not have to be continuously administered. The dosage form of the invention may be any form as long as the form is subcutaneous administration, and for example, subcutaneous administration by a healthcare professional and subcutaneous administration by self-injection are also included in the dosage form of the invention. In the case of continuous administration, the administration may be continued with the same dose as the last dose of the 16-week duration of administration, and the dose can also be appropriately increased or reduced. Moreover, in the case of continuous administration, the administration may be continued at the same dosage intervals as the last dosage interval of the 16-week duration of administration, and the dosage interval can also be appropriately adjusted. In the case of adjusting the dosage interval, the dosage interval can be further lengthened to three weeks or longer when the dosage interval has been two weeks, and the dosage interval can be further lengthened to five weeks or longer when the dosage interval has been four weeks. Examples of the dosage interval include 2 weeks, 3 weeks, 4 weeks, 5 weeks, 6 weeks, 7 weeks, 8 weeks, 10 weeks, 12 weeks, 14 weeks, 16 weeks and the like. In the case of continuous administration, the administration may be continued, for example, for 20 weeks, 22 weeks, 24 weeks, 34 weeks or longer. The dosage or the dosage interval can also be adjusted, for example by stopping the administration when sufficient efficacy is observed after administration for at least 16 weeks, decreasing the dosage or lengthening the dosage interval. Alternatively, even before 16 weeks, the dosage or the dosage interval can also be adjusted, for example by stopping the administration when sufficient efficacy is observed, decreasing the dosage or lengthening the dosage interval.

In the invention, the day on which the administration of the anti-OX40 antibody is started is counted as Day 1 (the initial day of the anti-OX40 antibody administration), and the day X of the anti-OX40 antibody administration (Day X) is counted from the initial day of the anti-OX40 antibody administration. In this regard, the day before the anti-OX40 antibody administration is counted as Day -1. The week in which the anti-OX40 antibody administration is started is counted as Week 0, and the Week Y is counted from the initial week of the anti-OX40 antibody administration. For example, the case where “the anti-OX40 antibody is continuously administered every two weeks for six weeks” or where “the anti-OX40 antibody is continuously administered every two weeks until the sixth week” means that the initial administration of the anti-OX40 antibody is on day 1 of Week 0 and that the second administration, the third administration and the fourth administration are on day 15 of Week 2, on day 29 of Week 4 and on day 43 of Week 6, respectively.

In some embodiments of the present disclosure, the administration is continued for at least 20 weeks, 22 weeks, 24 weeks or 34 weeks after starting the administration. In some embodiments, the administration of the anti-OX40 antibody is continued also after 16 weeks. In some embodiments, the administration of the anti-OX40 antibody or composition comprising the anti-OX40 antibody is continued for at least 20 weeks, 22 weeks, 24 weeks, 26 weeks, 28 weeks, 30 weeks, 32, weeks, 34 weeks, 36 weeks, 38 weeks, 40 weeks, 42 weeks, 44 weeks, 46 weeks, 48 weeks, 50 weeks, 52 weeks, 54 weeks, 56 weeks, 58 weeks, 60 weeks, 62 weeks, or 64 weeks after starting the administration.

In some embodiments, the anti-OX40 antibody is subcutaneously administered once in two weeks, three weeks or four weeks. In another embodiment, the antibody of the present invention is administrated subcutaneously at multiple doses. In particular, the anti-OX40 antibody or the composition comprising the anti-OX40 antibody is administered once a week, once in two weeks, once in three weeks, or once in four weeks for at least two weeks, for at least 4 weeks, for at least 6 weeks, for at least 8 weeks, for at least 10 weeks, for at least 12 weeks, for at least 14 weeks, for at least 16 weeks, for at least 18 weeks, for at least 20 weeks, for at least 22 weeks, for at least 24 weeks, for at least 26 weeks, for at least 28 weeks, for at least 30 weeks, for at least 32 weeks, for at least 34 weeks, for at least 36 weeks, for at least 38 weeks, for at least 40 weeks, for at least 42 weeks, for at least 44 weeks, for at least 46 weeks, for at least 48 weeks, for at least 50 weeks, for at least 52 weeks, for at least 54 weeks, for at least 56 weeks, or more. In some embodiments, the anti-OX40 antibody or the composition comprising the anti-OX40 antibody is administered once a week, once in two weeks, once in three weeks, or once in four weeks for at least 4 weeks, for at least 6 weeks, for at least 8 weeks, for at least 10 weeks, for at least 12 weeks, for at least 14 weeks, for at least 16 weeks, for at least 18 weeks, for at least 20 weeks, for at least 22 weeks, for at least 24 weeks, for at least 26 weeks, for at least 28 weeks, for at least 30 weeks, for at least 32 weeks, for at least 34 weeks, for at least 36 weeks, for at least 38 weeks, for at least 40 weeks, for at least 42 weeks, for at least 44 weeks, for at least 46 weeks, for at least 48 weeks, for at least 50 weeks, for at least 52 weeks, for at least 54 weeks, for at least 56 weeks, or more.

In a preferable embodiment, the present invention is a therapeutic method for an OX40-related immune- or allergy-related disease including subcutaneously administering an anti-OX40 antibody to a patient at a dose of 150 mg to 600 mg once in two weeks to four weeks continuously at the same dose.

In some embodiments of the present disclosure, wherein the dose is selected from 75, 100, 150, 200, 250, 300, 350, 400, 450, 500, 550 and 600 mg. In some embodiments of the present disclosure, the dose of the anti-OX40 antibody is selected from 75, 100, 150, 200, 250, 300, 350, 400, 450, 500, 550, 600, 650, 700, 750, 800, 850, 900, 950, and 1000 mg. In some embodiments, the dose of the anti-OX40 antibody is from about mg and about 2 g, or between about 100 mg and about 1.5 g, or between about 150 mg and about 1.2 g, or between about 150 mg and about 600 mg. More specifically the dose of the anti-OX40 antibody is at least 50 mg, or at least 60 mg, or at least 70 mg, or at least 80 mg, or at least 90 mg, or at least 100 mg, or at least 150 mg, or at least 200 mg, or at least 250 mg, or at least 300 mg, or at least 350 mg, or at least 400 mg, or at least 450 mg, or at least 500 mg, or at least 550 mg, or at least 600 mg, or at least 650 mg, or at least 700 mg, or at least 750 mg, or at least 800 mg, or at least 850 mg, or at least 900 mg, or at least 950 mg, or at least 1 g, or at least 1.2 g, or at least 1.5 g. The present disclosure also includes doses at any intermediate value between the above stated doses.

In another aspect, the anti-OX40 antibody is administered in a first dose comprising administering at least 50, 75, 100, 150, 200, 250, 300, 350, 400, 450, 500, 550, 600, 650, 700, 750, 800, 850, 900, 950, and 1000 mg once a week, once in two weeks, once in three weeks, or once in four weeks for at least two weeks, for at least 4 weeks, for at least 6 weeks, for at least 8 weeks, for at least 10 weeks, for at least 12 weeks, for at least 14 weeks, for at least 16 weeks, for at least 18 weeks, for at least 24 weeks, or more. In another aspect, the anti-OX40 antibody is administered in a first dose comprising from between about 50 mg and about 1 g, or between about 150 mg and about 800 mg, or between about 150 mg and about 600 mg, or between about 150 mg and about 300 mg once a week, once in two weeks, once in three weeks, or once in four weeks for at least 4 weeks, for at least 6 weeks, for at least 8 weeks, for at least 10 weeks, for at least 12 weeks, for at least 14 weeks, for at least 16 weeks, for at least 18 weeks, for at least 24 weeks, or more.

After the first dose, the anti-OX40 antibody may be administered at a second dose comprising at least 50, 75, 100, 150, 200, 250, 300, 350, 400, 450, 500, 550,600, 650, 700, 750, 800, 850, 900, 950, and 1000 mg once a week, once in two weeks, once in three weeks, or once in four weeks for at least two weeks, for at least 4 weeks, for at least 6 weeks, for at least 8 weeks, for at least 10 weeks, for at least 12 weeks, for at least 14 weeks, for at least 16 weeks, for at least 18 weeks, for at least 24 weeks, or more. After the first dose, the anti-OX40 antibody may be administered at a second dose comprising at least about 50 mg and about 1 g, or between about 150 mg and about 800 mg, or between about 150 mg and about 600 mg, or between about 150 mg and about 300 mg once a week, once in two weeks, once in three weeks, or once in four weeks for at least two weeks, for at least 4 weeks, for at least 6 weeks, for at least 8 weeks, for at least 10 weeks, for at least 12 weeks, for at least 14 weeks, for at least 16 weeks, for at least 18 weeks, for at least 24 weeks, or more.

### Pharmaceutical Compositions and Combination Therapies

The therapeutic method according to (1) which is combined with a known topical agent such as a steroid. In some embodiments, the therapeutic method is combined with a known topical agent. In some embodiments, the known topical agent is a steroid. In some embodiments, the both anti-OX40 antibody and a second agent selected from a corticosteroid or a calcineurin inhibitor are administered to the subject.

In another aspect, the anti-OX40 antibody is formulated as a pharmaceutical composition suitable for any one of the administration routes disclosed herein. In a preferred embodiment, the anti-OX40 antibody is formulated as a pharmaceutical composition suitable for subcutaneous administration. As used herein, the term “pharmaceutical composition” refers to one or more active agents formulated with a pharmaceutically acceptable carrier, excipient or diluent.

### Further Embodiments

Disclosed herein is an anti-OX40 antibody for use in the treatment of an OX40-related immune- or allergy-related disease, wherein the anti-OX40 antibody is a monoclonal antibody containing a heavy chain variable region (VH) containing the amino acid sequence of SEQ ID NO: 1 and a light chain variable region (VL) containing the amino acid sequence of SEQ ID NO: 2, and the anti-OX40 antibody is subcutaneously administered to a patient at a dose of 50 mg to 1000 mg once in two weeks to four weeks for at least 16 weeks.

In another aspect, the present disclosure provides the use of an anti-OX40 antibody for the manufacture of a pharmaceutical composition for treating an OX40-related immune- or allergy-related disease, wherein the anti-OX40 antibody is a monoclonal antibody containing a heavy chain variable region (VH) containing the amino acid sequence of SEQ ID NO: 1 and a light chain variable region (VL) containing the amino acid sequence of SEQ ID NO: 2, and the anti-OX40 antibody is subcutaneously administered to a patient at a dose of 50 mg to 1000 mg once in two weeks to four weeks for at least 16 weeks.

In another aspect, the present disclosure provides the use of an anti-OX40 antibody for the treatment of an OX40-related immune- or allergy-related disease, wherein the anti-OX40 antibody is a monoclonal antibody containing a heavy chain variable region (VH) containing the amino acid sequence of SEQ ID NO: 1 and a light chain variable region (VL) containing the amino acid sequence of SEQ ID NO: 2, and the anti-OX40 antibody is subcutaneously administered to a patient at a dose of 50 mg to 1000 mg once in two weeks to four weeks for at least 16 weeks.

In another aspect, the present disclosure provides a therapeutic method for an OX40-related immune- or allergy-related disease including subcutaneously administering an anti-OX40 antibody to a patient at a dose of 150 mg to 600 mg once in two weeks to four weeks continuously at the same dose. In some embodiments, the anti-OX40 antibody is a monoclonal antibody containing a heavy chain variable region (also called VH) containing the amino acid sequence of SEQ ID NO: 1 and a light chain variable region (also called VL) containing the amino acid sequence of SEQ ID NO: 2. In some embodiments, the administration is continued for at least 16 weeks, 20 weeks, 22 weeks, 24 weeks or 34 weeks after starting the administration. In some embodiments, the OX40-related immune- or allergy-related disease is atopic dermatitis.

In some embodiments, the anti-OX40 antibody is subcutaneously administered once in two weeks, three weeks or four weeks.

In some embodiments, the dose is selected from 150 mg, 300 mg, 450 mg and 600 mg.

In some embodiments, the OX40-related immune- or allergy-related disease is moderate to severe atopic dermatitis.

In some embodiments, the OX40-related immune- or allergy-related disease is moderate to severe atopic dermatitis which is poorly controllable using a topical agent or moderate to severe atopic dermatitis for which a topical therapy is not medically recommended. In some embodiments, the therapeutic method is combined with a known topical agent such as a steroid.

In some embodiments, the anti-OX40 antibody is KHK4083.

In another aspect, the present disclosure provides an anti-OX40 antibody for use in the treatment of an OX40-related immune- or allergy-related disease, wherein the anti-OX40 antibody is a monoclonal antibody containing a heavy chain variable region (VH) containing the amino acid sequence of SEQ ID NO: 1 and a light chain variable region (VL) containing the amino acid sequence of SEQ ID NO: 2, and the anti-OX40 antibody is subcutaneously administered to a patient at a dose of 50 mg to 1000 mg once in two weeks to four weeks continuously at the same dose.

In another aspect, the present disclosure provide use of an anti-OX40 antibody for the manufacture of a pharmaceutical composition for treating an OX40-related immune- or allergy-related disease, wherein the anti-OX40 antibody is a monoclonal antibody containing a heavy chain variable region (VH) containing the amino acid sequence of SEQ ID NO: 1 and a light chain variable region (VL) containing the amino acid sequence of SEQ ID NO: 2, and the anti-OX40 antibody is subcutaneously administered to a patient at a dose of 50 mg to 1000 mg once in two weeks to four weeks continuously at the same dose.

In some embodiments, the present disclosure provides use of an anti-OX40 antibody for the treatment of an OX40-related immune- or allergy-related disease, wherein the anti-OX40 antibody is a monoclonal antibody containing a heavy chain variable region (VH) containing the amino acid sequence of SEQ ID NO: 1 and a light chain variable region (VL) containing the amino acid sequence of SEQ ID NO: 2, and the anti-OX40 antibody is subcutaneously administered to a patient at a dose of 50 mg to 1000 mg once in two weeks to four weeks continuously at the same dose.

### EXAMPLES

The invention is explained in detail below with an Example, but the invention is not limited by the Example.

### Example 1

A phase II, global, double-blind, placebo-controlled, parallel-group trial was conducted according to the following protocol in patients with moderate to severe atopic dermatitis (AD) which is poorly controllable using a topical agent and moderate to severe AD patients to whom topical therapies are not medically recommended.

TABLE A-1

| Primary Objective | Endpoint |
|---|---|
| The efficacy of KHK4083 with four | Percentage change from |
| kinds of dosage and administration/four | baseline in EASI at Week 16 |
| doses is assessed by comparing the | |
| changes from baseline in the Eczema | |
| Area and Severity Index (EASI) with that | |
| of a placebo after 16-week repeated | |
| subcutaneous (SC) administration in | |
| moderate to severe AD patients. | |

TABLE A-2

| Secondary Objectives | Endpoints |
|---|---|
| Effects of 16-week repeated SC | Achievement of 50%, 75% and 90% or |
| administration of KHK4083 on skin | greater reductions from baseline in EASI |
| manifestation in moderate to severe AD | (EASI-50, EASI-75 and EASI-90) at |
| patients are compared with the effects of | Week 16 |
| a placebo. | Absolute change from baseline in EASI at |
| | Week 16 |
| | Absolute change and percentage change |
| | from baseline in Severity SCORing of |
| | Atopic Dermatitis (SCORAD) at Week 16 |
| | Achievement of score 0 or 1 and at least a |
| | 2-point reduction from baseline in |
| | Investigator's Global Assessment (IGA) of |
| | severity of skin manifestation at Week 16 |
| | Absolute change from baseline in Body |
| | Surface Area (BSA) of AD condition at |
| | Week 16 |
| Effects of 16-week repeated SC | Absolute change and percentage change |
| administration of KHK4083 on pruritus | from baseline in pruritus Numerical |
| and sleep in moderate to severe AD | Rating Scale (NRS) at Week 16 |
| patients are compared with the effects of | Absolute change and percentage change |
| a placebo. | from baseline in sleep disturbance NRS at |
| | Week 16 |
| Effects of 16-week repeated SC | Absolute change from baseline in |
| administration of KHK4083 on quality of | questionnaire on skin condition |
| life (QoL) in moderate to severe AD | (Dermatology Life Quality Index: DLQI) |
| patients are compared with the effects of | at Week 16 |
| a placebo. | |

TABLE A-3

| Secondary Objectives | Endpoints |
|---|---|
| Effects of 36-week repeated SC | Absolute change and percentage change |
| administration of KHK4083 on skin | from baseline in EASI at each assessment |
| manifestation in moderate to severe AD | time point |
| patients are examined. | Achievement of EASI-50, EASI-75 and |
| | EASI-90 at each assessment time point |
| | Absolute change and percentage change |
| | from baseline in SCORAD at each |
| | assessment time point |
| | Achievement of IGA score 0 or 1 and at |
| | least a 2-point reduction from baseline at |
| | each assessment time point |
| | Absolute change from baseline in BSA at |
| | each assessment time point |
| Effects of 36-week repeated SC | Absolute change and percentage change |
| administration of KHK4083 on pruritus | from baseline in pruritus NRS at each |
| and sleep in moderate to severe AD | assessment time point |
| patients are examined. | Absolute change and percentage change |
| | from baseline in sleep disturbance NRS at |
| | each assessment time point |
| Effects of 36-week repeated SC | Absolute change from baseline in DLQI at |
| administration of KHK4083 on QoL in | each assessment time point |
| moderate to severe AD patients are | |
| examined. | |

TABLE A-4

| Safety | Endpoints |
|---|---|
| Safety of repeated SC administration of | Adverse events (TEAEs) |
| KHK4083 in moderate to severe AD | Clinical test values |
| patients is examined. | Vital signs |
| | Standard 12-lead |
| | electrocardiogram |

TABLE A-5

| Exploratory Objectives | Endpoints |
|---|---|
| Pharmacokinetics and immunogenicity | Pharmacokinetics |
| of repeated SC administration of | Serum KHK4083 concentration |
| KHK4083 in moderate to severe AD | Pharmacokinetics parameters |
| patients are examined. | (Cₘₐₓ, C_{trough} and the like) |
| | Anti-KHK4083 antibody |
| Pharmacodynamic assessment of | Pharmacodynamic assessment |
| repeated SC administration of | Serum condition markers |
| KHK4083 in moderate to severe AD | (Thymus and activation- |
| patients is conducted. | regulated chemokine [TARC] |
| | and total serum IgE) |

### Subject Patients

Patients with moderate to severe AD which is poorly controllable using a topical agent or moderate to severe AD patients to whom topical therapies are not medically recommended. In this regard, to allow assessment of efficacy and safety in subjects who have not used any biopharmaceutical, the proportion of enrolled patients with medical records with a biopharmaceutical for the purpose of AD treatment is 50% or less of all the enrolled subjects.

### Criteria for Patients Selection

Patients selected by the following eligibility criteria were included in the clinical trial:
Patients who voluntarily signed written informed consent to participate in the trial.
Male or female patients who are 18 years of age or older at the time of consent.
Patients diagnosed with AD according to the American Academy of Dermatology Consensus Criteria (Eichenfield et al, 2014) or the local diagnostic criteria for 1 year or more prior to screening.
Patients with an EASI score of 16 or higher at screening and baseline.
Patients with an IGA score of “3: moderate” or higher at both screening and baseline.
Patients with BSA of 10% or more at both screening and baseline.
Patients who have been judged to show inadequate response to treatment with topical agents or it has been confirmed that topical therapies are not medically recommended because of important side effects or safety risks within 1 year prior to screening.
The inadequate response is defined as a state of not being able to achieve and maintain remission or low disease activity (corresponding to IGA=0 (without lesion) to 2 (mild)) even after application of at least a moderate-intensity topical corticosteroid (a calcineurin inhibitor (topical agent) is co-administered when necessary) for at least 28 days (or the maximum period recommended by the prescribing information of the product when the maximum period is shorter (for example, for 14 days in the case of a strongest-class topical corticosteroid)).
Patients who have history of AD treatment with systemic therapy within 1 year are also regarded to show inadequate response to a topical therapy and can be administered with KHK4083 after appropriate wash-out treatment.
The important side effects or the safety risks indicate cases which are stronger than the benefits of the treatment and are cases in which the investigator, a subinvestigator or the attending physician of the patient observes intolerance to the treatment, hyper-sensitivity reaction, severe skin atrophy or intolerance due to systemic conditions.
Women of childbearing potential and men of reproductive potential must agree to use highly effective contraceptive methods according to the guidance approved in each country from the time of consent to six months after the end of the study drug administration (for women) or from the start of the study drug administration to six months after the end of the study drug administration (for men). Female patients of childbearing potential must show negative results in a serum pregnancy test at screening and show negative results in a pregnancy test at baseline.
In the U.S. and Canada, women of childbearing potential who may have sexual intercourse with a male partner who has received contraception by a non-surgical method must agree to select and must conduct one of the following highly effective contraceptive methods (Clinical Trials Facilitation Group, 2014) from the time of written consent to six months after the final administration of the study drug.
Use of an oral, injection, transdermal or implanted estrogen-progestogen combined hormonal contraceptive should be settled (at least two months prior to the screening date). Subjects who use such a method less than two months prior to the screening date are required to use any one of the methods described in b) and c) until the hormonal contraceptive is settled.
Double barrier contraceptives: Use of pessary (cap or cervical/vaginal fornix cap) with spermicidal foam/gel/film/cream/suppository. In a country in which spermicidal condoms are not permitted, general condoms can be used with spermicidal cream. A female condom and a male condom should not be used together because either one or both of the products may break due to the friction between the condoms. The investigator or a subinvestigator determines an appropriate procedure with the subject according to the standard therapy in the country where the study drug is administered.
Intrauterine device or intrauterine contraceptive system
In Germany, women of childbearing potential and men of reproductive potential must agree to use a very effective contraceptive method that can achieve a failure rate of less than 1% per year from the time of consent to six months after the final administration of the study drug (for women) or from the start of the study drug administration to six months after the final administration of the study drug (for men). In this regard, women of childbearing potential must show negative results in a serum pregnancy test at screening and show negative results in urine pregnancy tests conducted at baseline and at dosage intervals. Contraceptive methods which are very effective when used correctly are listed below.
   Combined (estrogen and progestogen containing) hormonal contraception associated with inhibition of ovulation
   Oral administration
   Intravaginal administration
   Transdermal administration
   Progestogen-only hormonal contraception associated with inhibition of ovulation
   Oral administration
   Injection
   Implantation
   Contraceptive device
   Intrauterine hormone-releasing system
   Tubal ligation
   Vasectomized partner
   Sexual abstinence (Sexual abstinence is considered a very effective method only when defined as refraining from heterosexual intercourse during the entire period of the trial.)

### Supplement:

Women of childbearing potential do not include women who have received a

permanent sterilization method, postmenopausal women (no menses for 12 months or longer without an alternative medical cause (or according to the local postmenopausal standard)) and women without childbearing potential due to an anatomical reason.

Exclusion Criteria. The Following Patients Were Excluded From This Clinical Trial:
Patients having a severe complication which the investigator or a subinvestigator has judged to affect the conduct of the trial and the assessments. The complications include the following complications but are not limited to these complications: severe cardiovascular disease (e.g., class III or IV in New York Heart Association classification), poorly controlled diabetes (HbA1c of 9% or more), liver disease (e.g., class B or C in Child-Pugh classification), kidney disease, respiratory diseases, blood diseases, central nervous system diseases, mental disorders, autoimmune diseases and the like.
Patients having any of the following abnormal clinical test values at screening
   Serum creatinine: exceeding 1.5 mg/dL
   AST or ALT: at least 2.5 times the upper limit of the normal value
   Neutrophil count: less than 1.5×10³/micro L
   Other abnormal clinical test values which the investigator or subinvestigators consider to affect the completion or the assessment of this trial
Patients with active malignancy or patients with onset or history of treatment of malignancy within 5 years prior to consent (excluding curatively treated intraepithelial carcinoma of uterine cervix, cutaneous basal cell carcinoma and cutaneous squamous carcinoma).
Patients with medical history of alcohol or drug abuse within 1 year prior to the screening date or patients with alcoholism or drug addiction.
Patients who had or has any suicidal behavior.
Patients with medical history of severe immunoreaction (serum sickness, anaphylaxis or anaphylaxis-like reaction) to other biopharmaceuticals or any of the additives of KHK4083.
Patients who caught infections which require systemic administration (excluding oral administration) of an antibacterial agent, an antifungal agent, an antiviral agent or the like three times or more within 1 year prior to the baseline date.
Patients who caught an active chronic or acute infection which requires treatment by systemic administration of an antibiotic, an antiviral agent, an antiparasitic agent, an antiprotozoal agent or an antifungal agent within 4 weeks prior to the baseline date or caught an infection of the skin surface within 2 weeks prior to the baseline date.
Patients who received live vaccine (BCG, polio, measles, rubella or the like) administration within 12 weeks prior to the baseline date. Immunization with inactivated vaccines (hepatitis virus, pneumococcus, meningococcus, tetanus, diphtheria toxoid, acellular pertussis, inactivated polio, human papillomavirus, influenza vaccines excluding transnasal influenza vaccines and the like) is permitted.
Patients who received administration of a biopharmaceutical (including the study drug) within 12 weeks (16 weeks in Japan) or five times the half-life (longer one) prior to the baseline date.
Patients who used three biopharmaceuticals or more (including the study drug) within 2 years prior to the baseline date.
Patients who participated in a clinical trial of a drug or an equivalent study within 4 weeks (16 weeks in Japan) or five times the half-life (longer one) prior to the baseline date and to whom the study drug (excluding biopharmaceuticals) was administered or a non-approved medical device was used.
Patients who received administration of any of the drugs or any of the therapies below within 4 weeks or five times the half-life (longer one) prior to the baseline date:
   Systemic administration of a corticosteroid (Combined inhalation, eye drops, ear drops or nasal drops is permitted, but combined suppository or enema containing a corticosteroid is prohibited.)
   Systemic administration of methotrexate, ciclosporin, mycophenolic acid, tacrolimus, thalidomide or other immunosuppressive agents
   Phototherapy (psoralen-UV (PUVA) therapy, ultraviolet B (UVB) therapy, narrow-band UVB therapy, ultraviolet Al (UVA1) therapy, excimer light and the like) for the purpose of AD treatment
   JAK inhibitors
Patients who received administration of any of the drugs or any of the therapies below for the purpose of AD treatment within 1 week prior to the baseline date:
   Topical corticosteroid agents
   Calcineurin inhibitors or other immunosuppressive agents (topical)
   Topical agents such as crotamiton and crisaborole (Eucrisa(R))
   Topical agent mixtures containing a corticosteroid, a calcineurin inhibitor or another immunosuppressive agent
   Herb medicines (Jumihaidokuto, Shofusan, Saikoseikanto, Hochuekkito and the like)
Patients with planned surgical treatment or invasive procedure during the participation in the trial (for example, dental implantation or nonemergency minimally invasive heart surgery).
Patients who cannot stop any prohibited concomitant medication or prohibited concomitant therapy.
Patients who are pregnant or nursing or female patients who desire for childbearing.
Patients infected with human immunodeficiency virus (HIV) or with a positive HIV antibody test as a result of screening. Patients with acquired, common variable or inherited, primary or secondary immunodeficiency.
Patients with active hepatitis B (HB) infection under any of the following conditions as a result of screening:
   Hepatitis B surface antigen (HBs antigen) positive
   Hepatitis B core antibody (HBc antibody) positive or hepatitis B virus (HBV)-DNA positive
   In Japan, HBc antibody and/or HBs antibody positive and HBV-DNA positive.

Here, active antibody positive patients due to hepatitis B vaccination who are not infected with hepatitis B at the time of screening are not required for HBV-DNA measurement and are permitted to enroll in this test.

However, when any of the results is uncertain or when the results cannot be determined, alternative testing determined in each region should be used for confirmation.
Patients who are hepatitis C virus (HCV) antibody positive as a result of screening and who are confirmed to be infected with HCV by RNA or another testing. For uncertain results, alternative testing determined in each region should be used for confirmation.
Patients with sign or history of treated or untreated active tuberculosis or patients with latent tuberculosis (defined as having no proof of clinically evident active tuberculosis and being positive in a Purified Protein Derivative (PPD) test or an interferon gamma release assay (IGRA)) who have completed the treatment at least 12 months prior to the baseline date or who are not treated. Assessment of tuberculosis is in accordance with the local therapy standard or as defined by the local guideline, which includes a PPD or IGRA test and may include medical history, physical examination and chest radiography. Latent tuberculosis patients who satisfy any of the following conditions are permitted to enroll.
Patients who completed appropriate antituberculosis treatment in accordance with the local guideline or therapy standard within 12 months prior to the baseline date.
Patients receiving appropriate antituberculosis treatment (for example, isoniazid) in accordance with the local guideline or therapy standard at least for 28 days (21 days in Japan) prior to the baseline date.
Patients who have participated in a KHK4083 trial and received the study drug.
Other patients who are judged by the investigator or a subinvestigator to be not suitable for participation in this trial.

### Trial Design

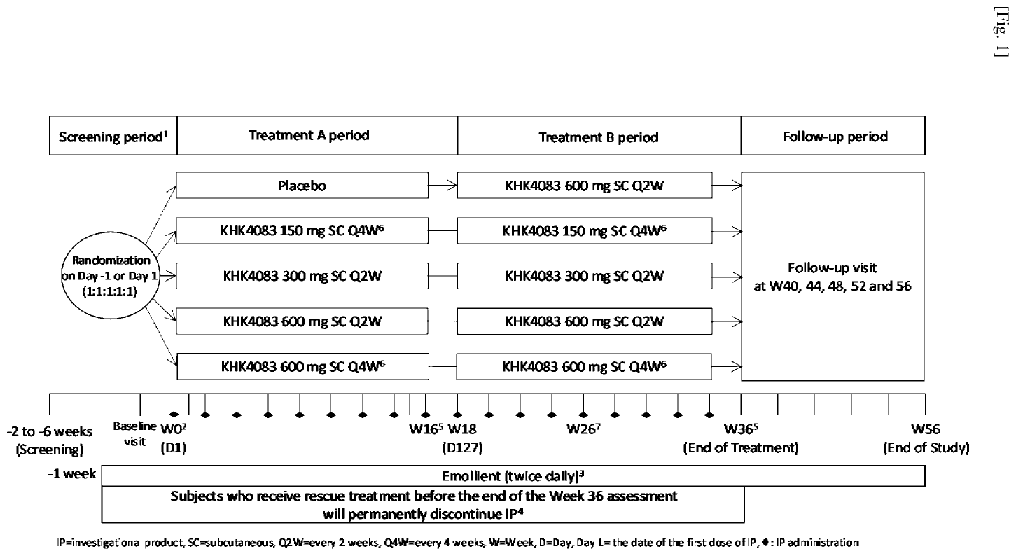

This trial includes at least a 2-week (maximum 6-week) screening period, an 18-week placebo or study drug administration period (Treatment A), a subsequent 18-week study drug administration period (Treatment B) and a 20-week follow-up period (FOLLOW-UP) (FIG. 1). The subjects receive repeated SC administration of a placebo or the study drug every two weeks under a double-blind condition (Week 0 (Day 1), Weeks 2, 4, 6, 8, 10, 12, 14, 16, 18, 20, 22, 24, 26, 28, 30, 32 and 34). The final administration is at Week 34. Then, the follow-up is at Weeks 40, 44, 48, 52 and 56.

The primary endpoint was assessed by the absolute change from baseline (the value at Week 0) in EASI scores at Week 16. Here, Q4W below means the administration schedule of once in four weeks, and Q2W means the administration schedule of once in two weeks. The screening period is a period in which the eligibility for this trial is judged without the administration of KHK4083.

### Treatment A Period (From Week 0 to Before Study Drug Administration at Week 18)

All the subjects who were confirmed to satisfy the selection criteria and not to meet any of the exclusion criteria during the screening period are randomly assigned to a placebo group, a KHK4083 150 mg Q4W group, a 300 mg Q2W group, a 600 mg Q2W group and a 600 mg Q4W group in a 1:1:1:1:1 ratio. The subjects receive repeated SC administration of the study drug every two weeks under a double-blind condition (the final administration is at Week 16). In the 150 mg Q4W group and the 600 mg Q4W group, the placebo is administered between the KHK4083 administrations at four-week intervals, and the study drug and the placebo are administered alternately every two weeks to ensure the blindability.

### Treatment B Period (From Study Drug Administration at Week 18 to Week 36, Final Administration at Week 34)

KHK4083 is administered to all the subjects from Week 18 under a double-blind condition (the final administration is at Week 34). In the Treatment B Period, the subjects randomized in the placebo group in the Treatment A period receive repeated SC administration of 600 mg of KHK4083 once in two weeks from Week 18. The subjects randomized in the KHK4083 groups in the Treatment A Period receive continuous administration of KHK4083 at the same dosages at the same dosage intervals as those in the Treatment A Period.

“When the results of IGA at Week 26 deteriorate or do not differ from the baseline value (the value at Week 0) and when the IGA at Week 26 does not differ or deteriorates from the IGA at Week 18”, the study drug administration to the subject at Week 26 is stopped, and the trial is terminated after the checkup at the end.

### Follow-Up Period

The period after the completion of the scheduled checkup at Week 36 is a follow-up period, and the follow-up is every four weeks until Week 56. The subjects who receive rescue treatment at Week 36 and later undergo the trial until the end of the trial at Week 56 rather than terminating this trial.

### Flow Cytometry

Blood samples for flow cytometry are collected from the subjects who voluntarily agree. When the timing overlaps the study drug administration, the sample is taken before the study drug administration. The OX40-positive cells and the CLA-positive memory T cells in the blood of the patients are counted. When OX40-positive cells in the blood are analyzed, OX40-positive cells in helper T cells are analyzed by using helper T cell markers. Therefore, OX40-positive cells in the blood herein means OX40 positive helper T cells in the blood.

Timings: at screening, at baseline and at Weeks 1, 8, 16, 36, 40, 44, 48 and 52. See FIG. 1 showing a summary of the trial design.

### Results

### Enrolled Subjects

In this trial, 274 subjects who satisfied the eligibility criteria were assigned to the KHK4083 and placebo groups. For the efficacy assessment items (endpoints), the FAS (Full Analysis Set) was the major population for the analysis, and the population excluding the randomized subjects who met any of the following conditions was the FAS:

### Subjects Who Have Never Received the Study Drug Administration

### Subjects Without Any of the EASI Scores After Starting the Study Drug Administration Until Week 16

The population for the FAS analysis in this trial included 267 subjects. The results of the primary endpoints and the secondary endpoints shown below are the data of the population for the FAS analysis.

### Details of Populations for Analyses:

TABLE 1

| | KHK4083 | KHK4083 | KHK4083 | KHK4083 | | |
|---|---|---|---|---|---|---|
| | 150 mg | 600 mg | 300 mg | 600 mg | | |
| | Q4W | Q4W | Q2W | Q2W | Placebo | Total |
| | N = 54 | N = 54 | N = 55 | N = 54 | N = 57 | N = 274 |
| Variable | n (%) | n (%) | n (%) | n (%) | n (%) | n (%) |
| FAS | 52(96.3) | 52(96.3) | 52(94.5) | 54(100) | 57(100) | 267(97.4) |
| PPS | — | — | — | — | — | — |
| SS | 54(100) | 53(98.1) | 55(100) | 54(100) | 57(100) | 273(99.6) |
| PKS | 53(98.1) | 53(98.1) | 54(98.2) | 54(100) | 0 | 214(78.1) |

FAS = Full Analysis Set,
PKS = Pharmacokinetic Analysis Set,
PPS = Per Protocol Set,
SS = Safety Analysis Set.

### Primary Endpoint

As the primary endpoint, “the percentage change from baseline in EASI scores at Week 16” was assessed. The results of the administration groups, the KHK4083 150 mg Q4W group, the KHK4083 600 mg Q4W group, the KHK4083 300 mg Q2W group, the KHK4083 600 mg Q2W group and the placebo group, were 48.3%, 49.68%, 61.05%, 57.63% and 14.98%, respectively, and efficacy compared to the placebo group was confirmed with a statistically significant difference (p<0.001) in all the KHK4083 administration groups (Table 2).

Percentage Changes from Baseline in EASI Scores at Week 16

TABLE 2

| | KHK4083 | KHK4083 | KHK4083 | KHK4083 | |
|---|---|---|---|---|---|
| | 150 mg | 600 mg | 300 mg | 600 mg | |
| | Q4W | Q4W | Q2W | Q2W | Placebo |
| Statistics | N = 52 | N = 52 | N = 52 | N = 54 | N = 57 |
| LS Mean | −48.30 | −49.68 | −61.05 | −57.63 | −14.98 |
| 95% CI | (−62.60, −34.00) | (−64.25, −35.12) | (−75.17, −46.92) | (−71.56, −43.70) | (−28.57, −1.38) |
| LS Mean of Difference vs Placebo | −33.33 | −34.71 | −46.07 | −42.65 | |
| 95% CI of Difference vs Placebo | (−51.31, −15.34) | (−52.67, −16.74) | (−63.98, −28.16) | (−60.35, −24.96) | |
| P value | <0.001 | <0.001 | <0.001 | <0.001 | |

### Secondary Endpoints

As the major secondary endpoints, “the proportions of the subjects with a 50%, 75% and 90% or greater reduction from baseline in EASI scores (EASI-50, EASI-75 and EASI-90) at Week 16”, “the proportion of the subjects whose IGA scores were 0 or 1 with at least a 2-point reduction from baseline at Week 16” and “the proportion of the subjects with at least a 4-point reduction from baseline in pruritus NRS scores at Week 16” were assessed.

In all the endpoints, all the KHK4083 administration groups showed higher efficacy than those of the placebo group (Table 3).

In the clinical trial for patients with moderate to severe atopic dermatitis, in “the proportion of the subjects with a 75% or greater reduction from baseline in EASI scores (EASI-75) at Week 16” and “the proportion of the subjects whose IGA was 0 or 1 with at least a 2-point reduction from baseline at Week 16” that are generally considered as the primary endpoints, all the KHK4083 administration groups showed higher efficacy compared to the placebo group with a statistically significant difference (Tables 4-7). Additionally, in “the proportion of the subjects with at least a 4-point reduction from baseline in pruritus NRS scores at Week 16”, the KHK4083 600 mg Q4W group, the KHK4083 300 mg Q2W group and the KHK4083 600 mg Q2W group showed higher efficacy compared to the placebo group with a statistically significant difference.

### Proportions of Achieved for Secondary Endpoints at Week 16 (%):

TABLE 3

| | | | | | Placebo/ |
|---|---|---|---|---|---|
| | KHK4083 | KHK4083 | KHK4083 | KHK4083 | KHK4083 |
| | 150 mg | 600 mg | 300 mg | 600 mg | 600 mg |
| Variable | Q4W | Q4W | Q2W | Q2W | Q2W |
| Achievement of EASI-50 | 57.7 | 59.6 | 69.2 | 64.8 | 29.8 |
| Achievement of EASI-75 | 44.2 | 40.4 | 53.8 | 38.9 | 10.5 |
| Achievement of EASI-90 | 19.2 | 11.5 | 36.5 | 18.5 | 3.5 |
| Achievement of IGA Score of | 19.2 | 15.4 | 30.8 | 18.5 | 1.8 |
| 0 or 1 with reduction | | | | | |
| from Baseline of >=2 Point | | | | | |
| Achievement of >=4 Reduction | 36.5 | 46.2 | 55.8 | 44.4 | 19.3 |
| in Pruritus NRS score | | | | | |

### Proportions of Subjects With 50% or Greater Reduction From Baseline in EASI Scores (EASI-50) at Week 16:

TABLE 4
Variable: EASI-50

| | KHK4083 | KHK4083 | KHK4083 | KHK4083 | |
|---|---|---|---|---|---|
| | 150 mg | 600 mg | 300 mg | 600 mg | |
| | Q4W | Q4W | Q2W | Q2W | Placebo |
| Statistics | N = 52 | N = 52 | N = 52 | N = 54 | N = 57 |
| n (%) | 30 (57.7) | 31 (59.6) | 36 (69.2) | 35 (64.8) | 17 (29.8) |
| Exact 95% CI (%) | (43.20, 71.27) | (45.10, 72.99) | (54.90, 81.28) | (50.62, 77.32) | (18.43, 43.40) |
| Difference vs Placebo (%) | 27.87 | 29.79 | 39.41 | 34.99 | |
| Exact 95% CI of | (8.97, 45.13) | (10.94, 46.94) | (20.96, 55.77) | (16.35, 51.69) | |
| Difference vs Placebo (%) | | | | | |
| P value of Difference vs | 0.003 | 0.002 | <0.001 | <0.001 | |
| Placebo | | | | | |

CI = confidence interval, EASI = Eczema Area and Severity Index, NRI = non-responder imputation.
Note:
EASI-50, EASI-75 and EASI-90 are defined as achievement of >=50%, >=75%, or >=90% reduction from baseline in EASI scores respectively.
Note:
P-value is calculated by Fisher’s exact test with no adjustment as explanatory purpose.
program location: /projects/khkco237307/stats/primary/prog/tables/t_sum_ac_easi_xx.sas
Final Analysis
CONFIDENTIAL
Output date: 2 Jul. 2021 12:49 GMT

### Proportions of Subjects With 75% or Greater Reduction From Baseline in EASI Scores (EASI-75) at Week 16:

TABLE 5
Variable: EASI-75

| | KHK4083 | KHK4083 | KHK4083 | KHK4083 | |
|---|---|---|---|---|---|
| | 150 mg | 600 mg | 300 mg | 600 mg | |
| | Q4W | Q4W | Q2W | Q2W | Placebo |
| Statistics | N = 52 | N = 52 | N = 52 | N = 54 | N = 57 |
| n (%) | 23 (44.2) | 21 (40.4) | 28 (53.8) | 21 (38.9) | 6 (10.5) |
| Exact 95% CI (%) | (30.47, 58.67) | (27.01, 54.90) | (39.47, 67.77) | (25.92, 53.12) | (3.96, 21.52) |
| Difference vs Placebo (%) | 33.70 | 29.86 | 43.32 | 28.36 | |
| Exact 95% CI of | (15.09, 50.51) | (11.10, 46.94) | (25.34, 59.21) | (10.02, 45.71) | |
| Difference VS Placebo (%) | | | | | |
| P value of Difference vs | <0.001 | <0.001 | <0.001 | <0.001 | |
| Placebo | | | | | |

CI = confidence interval, EASI = Eczema Area and Severity Index, NRI = non-responder imputation.
Note:
EASI-50, EASI-75 and EASI-90 are defined as achievement of >=50%, >=75%, or >=90% reduction from baseline in EASI scores respectively.
Note:
P-value is calculated by Fisher's exact test with no adjustment as explanatory purpose.
program location: /projects/khkco237307/stats/primary/prog/tables/t_sum_ac_easi_xx.sas
Final Analysis
CONFIDENTIAL
Output date: 2 Jul. 2021 12:49 GMT

### Proportions of Subjects With 90% or Greater Reduction From Baseline in EASI Scores (EASI-90) at Week 16:

TABLE 6
Variable: EASI-90

| | KHK4083 | KHK4083 | KHK4083 | KHK4083 | |
|---|---|---|---|---|---|
| | 150 mg | 600 mg | 300 mg | 600 mg | |
| | Q4W | Q4W | Q2W | Q2W | Placebo |
| Statistics | N = 52 | N = 52 | N = 52 | N = 54 | N = 57 |
| n (%) | 10 (19.2) | 6 (11.5) | 19 (36.5) | 10 (18.5) | 2 (3.5) |
| Exact 95% CI (%) | (9.63, 32.53) | (4.35, 23.44) | (23.62, 51.04) | (9.25, 31.43) | (0.43, 12.11) |
| Difference vs Placebo (%) | 15.72 | 8.03 | 33.03 | 15.01 | |
| Exact 95% CI of Difference vs | (−3.24, 33.81) | (−10.97, 26.39) | (14.45, 49.81) | (−4.05, 33.01) | |
| Placebo (%) | | | | | |
| P value of Difference vs Placebo | 0.012 | 0.148 | <0.001 | 0.013 | |

CI = confidence interval, EASI = Eczema Area and Severity Index, NRI = non-responder imputation.
Note:
EASI-50, EASI-75 and EASI-90 are defined as achievement of >=50%, >=75%, or >=90% reduction from baseline in EASI scores respectively.
Note:
P-value is calculated by Fisher's exact test with no adjustment as explanatory purpose.
program location: /projects/khkco237307/stats/primary/prog/tables/t_sum_ac_easi_xx.sas
Final Analysis
CONFIDENTIAL
Output date: 2 Jul. 2021 12:49 GMT

### Proportions of Subjects Whose IGA was 0 or 1 With at Least 2-Point Reduction From Baseline at Week 16:

TABLE 7
Variable: Achievement of IGA Score of 0 or 1 with a reduction from Baseline of >=2 Point

| | KHK4083 | KHK4083 | KHK4083 | KHK4083 | |
|---|---|---|---|---|---|
| | 150mg | 600mg | 300mg | 600mg | |
| | Q4W | Q4W | Q2W | Q2W | Placebo |
| Statistics | N = 52 | N = 52 | N = 52 | N = 54 | N = 57 |
| n (%) | 10 (19.2) | 8 (15.4) | 16 (30.8) | 10 (18.5) | 1 (1.8) |
| Exact 95% CI (%) | (9.63, 32.53) | (6.88, 28.08) | (18.72, 45.10) | (9.25, 31.43) | (0.04, 9.39) |
| Difference vs Placebo (%) | 17.48 | 13.63 | 29.01 | 16.76 | |
| Exact 95% CI of | (−1.51, 35.46) | (−5.39, 31.78) | (10.46, 46.21) | (−2.29, 34.63) | |
| Difference vs Placebo (%) | | | | | |
| P value of Difference | 0.002 | 0.013 | <0.001 | 0.003 | |
| vs Placebo | | | | | |

CI = confidence interval, EASI = Eczema Area and Severity Index, NRI = non-responder imputation.
Note:
EASI-50, EASI-75 and EASI-90 are defined as achievement of >=50%, >=75%, or >=90% reduction from baseline in EASI scores respectively.
Note:
P-value is calculated by Fisher's exact test with no adjustment as explanatory purpose.
program location: /projects/khkco237307/stats/primary/prog/tables/t_sum_ac_easi_xx.sas
Final Analysis
CONFIDENTIAL
Output date: 2 Jul. 2021 12:49 GMT

### Discussion

In this trial, the efficacy and the safety of KHK4083 which was administered at four types of dosage, namely 150 mg Q4W, 600 mg Q4W, 300 mg Q2W and 600 mg Q2W, were compared with those of the placebo. For the primary endpoint, efficacy compared to the placebo group was confirmed with a statistically significant difference in all the KHK4083 administration groups. Also for the major secondary endpoints, a similar tendency was observed. This shows that administration of 150 mg to 600 mg of KHK4083 once in two weeks to four weeks for at least 16 weeks exhibits excellent improvement effects in patients with moderate to severe atopic dermatitis which is poorly controllable using a topical agent or moderate to severe atopic dermatitis patients to whom topical therapies are not medically recommended. Furthermore, it was found that, when KHK4083 is administered continuously after administration for 16 weeks as an introduction period, the improvement effects of KHK4083 are further enhanced and that the improvement effects last for a long period even after the subsequent completion of the administration.

### Efficacy in Administration Groups After Week 16

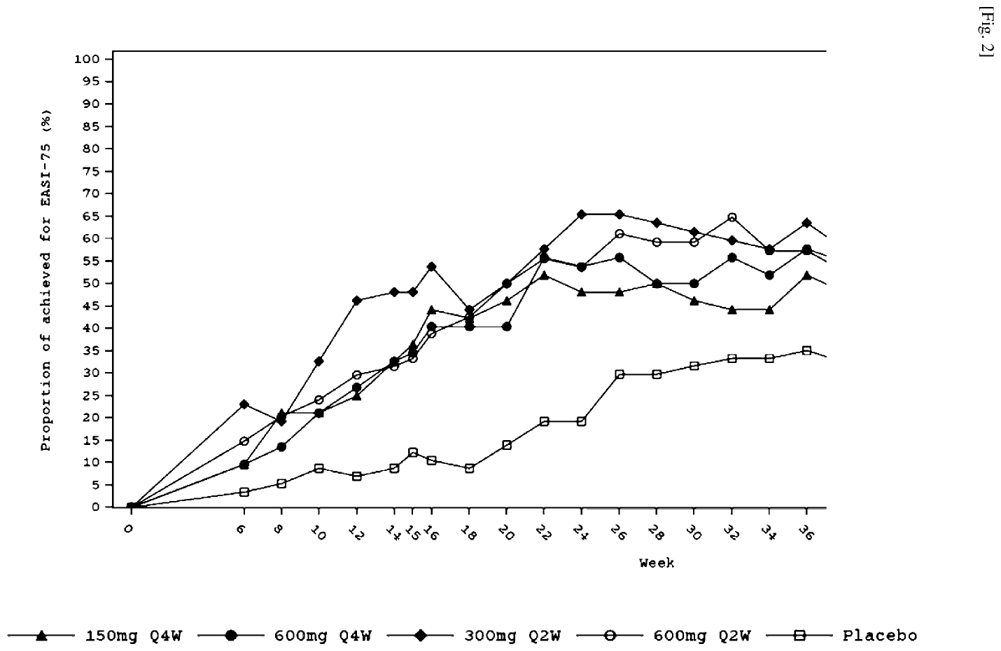

The changes with time of the proportion of achieved for EASI-75 (%) of the administration groups until Week 36 are shown in FIG. 2. In the placebo group, the actual drug was administered at Week 18 and later as in the 600 mg Q2W group. The highest proportions of achieved for EASI-75 were 51.9% at Week 22 in the 150 mg Q4W group, 57.7% at Week 36 in the 600 mg Q4W group, 65.4% at Week 24 in the 300 mg Q2W group and 64.8% at Week 32 in the 600 mg Q2W group.

The proportions of achieved for EASI-50, EASI-75, EASI-90 and IGA0/1 (%) of the administration groups at Week 16, Week 24 and Week 36 are shown in Table 8 below. In all the KHK4083 administration groups, the proportions of achieved for each endpoint at Week 16 were higher than those of the placebo group. Moreover, in all the KHK4083 administration groups, the proportions of achieved for EASI-75, EASI-90 and IGA0/1 at Week 24 and Week 36 were higher than those at Week 16.

### Proportion of Achievement for EASI and IGA at W16, W24 and W36:

TABLE 8

| | | 150 mg | 600 mg | 300 mg | 600 mg | |
|---|---|---|---|---|---|---|
| Assessment | | Q4W | Q4W | Q2W | Q2W | Placebo |
| Item | Week | (N = 54) | (N = 54) | (N = 54) | (N = 54) | (N = 57) |
| EASI-50 | W16 | 57.7 | 59.6 | 69.2 | 64.8 | 29.8 |
| (%) | W24 | 61.5 | 65.4 | 75.0 | 74.1 | |
| | W36 | 59.6 | 65.4 | 69.2 | 66.7 | |
| EASI-75 | W16 | 44.2 | 40.4 | 53.8 | 38.9 | 10.5 |
| (%) | W24 | 48.1 | 53.8 | 65.4 | 53.7 | |
| | W36 | 51.9 | 57.7 | 63.5 | 57.4 | |
| EASI-90 | W16 | 19.2 | 11.5 | 36.5 | 18.5 | 3.5 |
| (%) | W24 | 26.9 | 26.9 | 50.0 | 29.6 | |
| | W36 | 34.6 | 36.5 | 53.8 | 38.9 | |
| IGA 0/1 | W16 | 19.2 | 15.4 | 30.8 | 18.5 | 1.8 |
| (%) | W24 | 26.9 | 19.2 | 38.5 | 22.2 | |
| | W36 | 34.6 | 26.9 | 51.9 | 35.2 | |

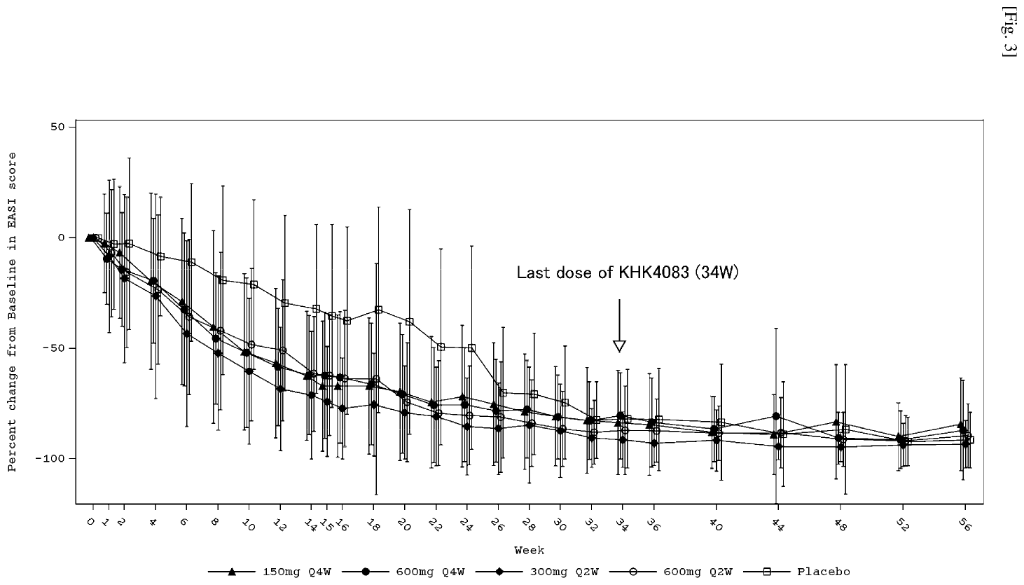

The percentage changes from baseline in EASI scores of the administration groups are shown in FIG. 3. In the placebo group, the actual drug was administered at Week 18 and later as in the 600 mg Q2W group. As shown in FIG. 3, in all the KHK4083 administration groups, the EASI scores improved from baseline. Surprisingly, the improvement effects lasted at least for 20 weeks until Week 56 after the final administration at Week 34.

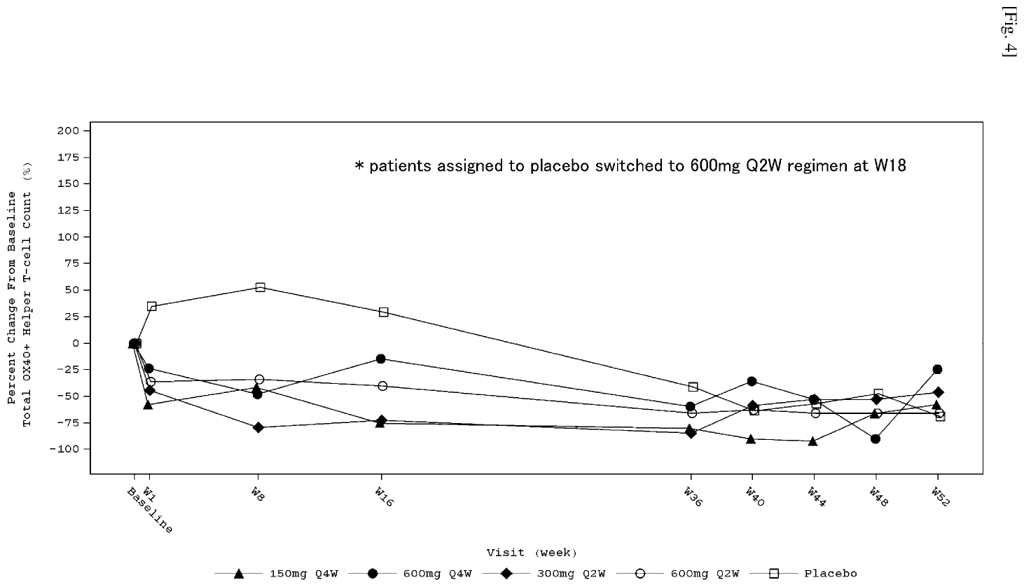

The percentage changes from baseline in the blood OX40-positive cell counts of the administration groups are shown in FIG. 4. From this figure, it was found that the blood OX40-positive cell counts decreased after the administration of KHK4083 in all the KHK4083 administration groups. The decrease lasted at least until Week 52 after the final administration at Week 34. The lasting decrease in the blood OX40-positive cell count is believed to contribute to the lasting efficacy of KHK4083.

The trial participation time and the administration start time are different depending on the patients, and therefore, the progress of the trial varies for each patient. The “Efficacy in Administration Groups after Week 16” described above was obtained on Oct. 26, 2020, which is represented by <A>. All the patients reached Week 36 at the time point of <A>, and data were all obtained (FIGS. 2 and Table 8), but in the data at the week thereafter, data of patients who reached the week and data of patients who did not reach the week at the time point of data analysis were mixed. The number of patients who reached the week at the time point of data analysis and were suitable as subjects for efficacy analysis is shown at each week from Week 1 to Week 56 in the Descriptive summary of EASI score at each scheduled visit in Tables 18-19 below.

The percentage changes in the EASI scores in FIG. 3 and the percentage changes in the blood OX40-positive cell counts in FIG. 4 were determined by analysis of only numerical values of patients having data at Week 40 and later. In the placebo group, the actual drug was administered in the same manner as in the 600 mg Q2W group at Week 18 and later, that is, in the placebo group, KHK4083 was administered at a dose of 600 mg once in two weeks at Week 18 and later.

In the proportions of patients who achieved EASI-75 in Tables 9-17, patients who did not reach the week at Week 40 and later or did not yet obtain data are counted as patients who did not achieve EASI-75.

### <B> Efficacy in Administration Groups After Week 16 (as of Feb. 1, 2021, Final Analysis Results Until Week 56)

After all the patients completed or terminated the trial, the same analysis as the above <A> was conducted.

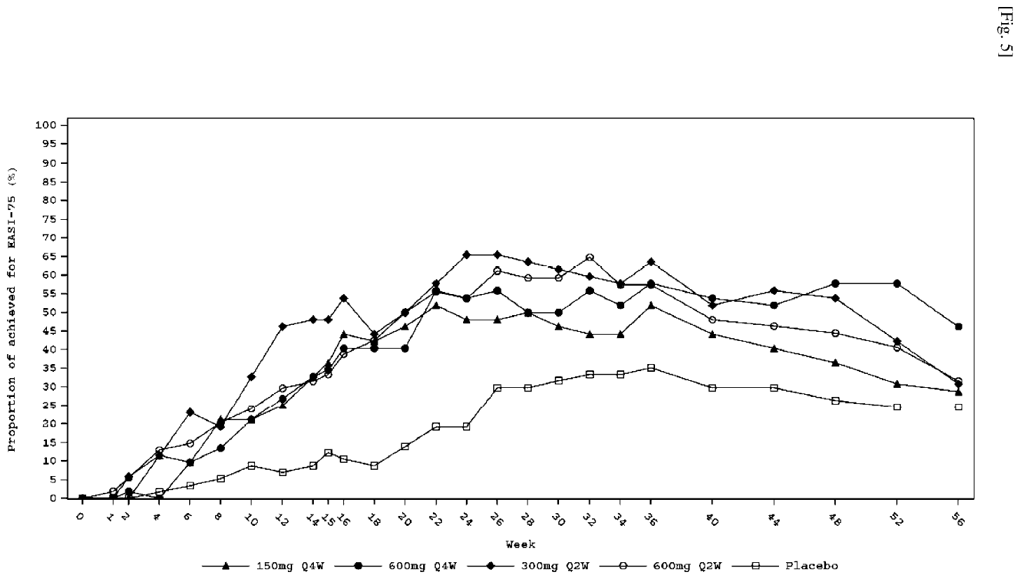

The changes with time of the proportion of achieved for EASI-75 (%) of the administration groups until Week 56 are shown in FIG. 5. In the placebo group, KHK4083 was administered at a dose of 600 mg once in two weeks at Week 18 and later. The highest proportions of achieved for EASI-75 were 51.9% at Weeks 22 and 36 in the 150 mg Q4W group, 57.7% at Weeks 36, 48, and 52 in the 600 mg Q4W group, 65.4% at Weeks 24 and 26 in the 300 mg Q2W group and 64.8% at Week 32 in the 600 mg Q2W group.

The proportions of achieved for EASI-50, EASI-75, EASI-90 and IGA0/1 (%) of the administration groups at Week 16, Week 24 and Week 36 did not differ from those of Table 8 for <A>. The numerical data of the proportion of achieved for EASI-50 (%), the proportion of achieved for EASI-75 (%), the proportion of achieved for EASI-90 (%) and the proportion of achieved for IGA0/1 (%) of the administration groups are shown in Tables 21-28, respectively. In all the KHK4083 administration groups, the proportions of achieved for each endpoint at Week 16 were higher than those of the placebo group. Moreover, in all the KHK4083 administration groups, the proportions of achieved for EASI-75, EASI-90 and IGA0/1 at Week 24 and Week 36 were higher than those at Week 16.

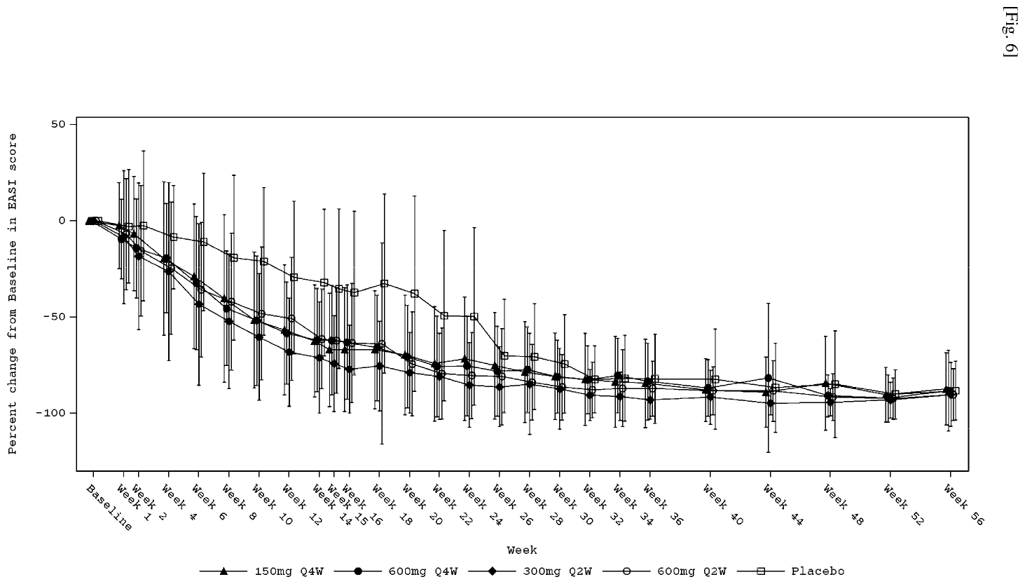

The percentage changes from baseline in EASI scores of the administration groups are shown in FIG. 6. The numerical data of FIG. 6 are shown in Tables 29-37. The error bar in FIG. 6 shows SD. In the placebo group, the KHK4083 was administered at a dose of 600 mg once in two weeks at Week 18 and later. As shown in FIG. 6 and Tables 29-37, the EASI scores improved from baseline in all the KHK4083 administration groups. Surprisingly, the improvement effect lasted for at least 22 weeks or more until Week 56 after the final administration at Week 32 or 34. Tables 40-47 show “percent change from baseline in EASI score at each scheduled visit without regard to prohibited concomitant medications (Full Analysis Set)”. In this analysis, the EASI data obtained after the start of prohibited concomitant medication was also included. On the other hand, tables 29-37 show “percent change from baseline in EASI score at each scheduled visit (Full Analysis Set)”. In this analysis, the EASI data obtained after the start of prohibited concomitant medications and therapies was deemed as missing data. There was no remarkable difference between these results.

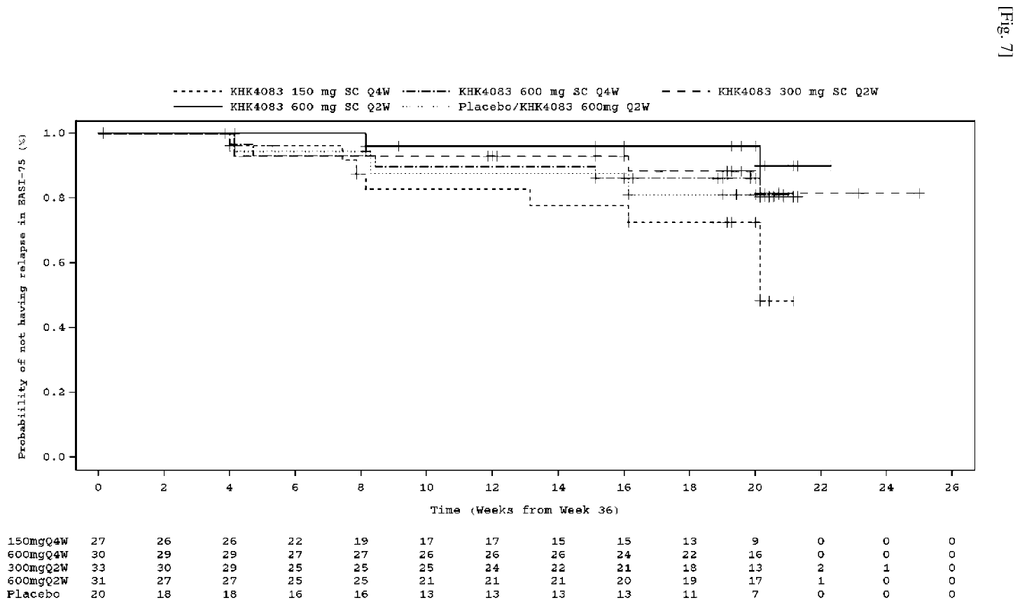

The time (weeks) taken until the patients who achieved EASI-75 at Week 36 relapse (loss of EASI-75) without administration of KHK4083 thereafter was analyzed by Kaplan-Meier method. The results are shown in a graph in FIG. 7, and by numerical data in Table 38. The numerical value on the lower side of FIG. 7 indicates the number of patients to be assessed at the week. FIG. 7 and Table 38 showed that many subjects maintained EASI-75 over a long period of time until Week 56 after the final administration of the actual drug at Week 32 or 34 in the 600 mg Q4W group, the 300 mg Q2W group and the 600 mg Q2W group.

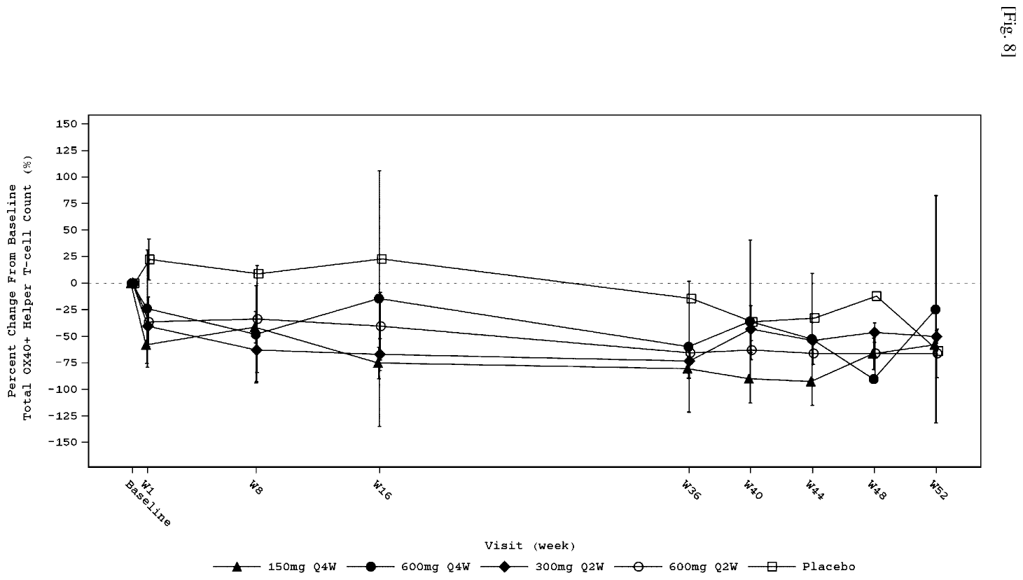

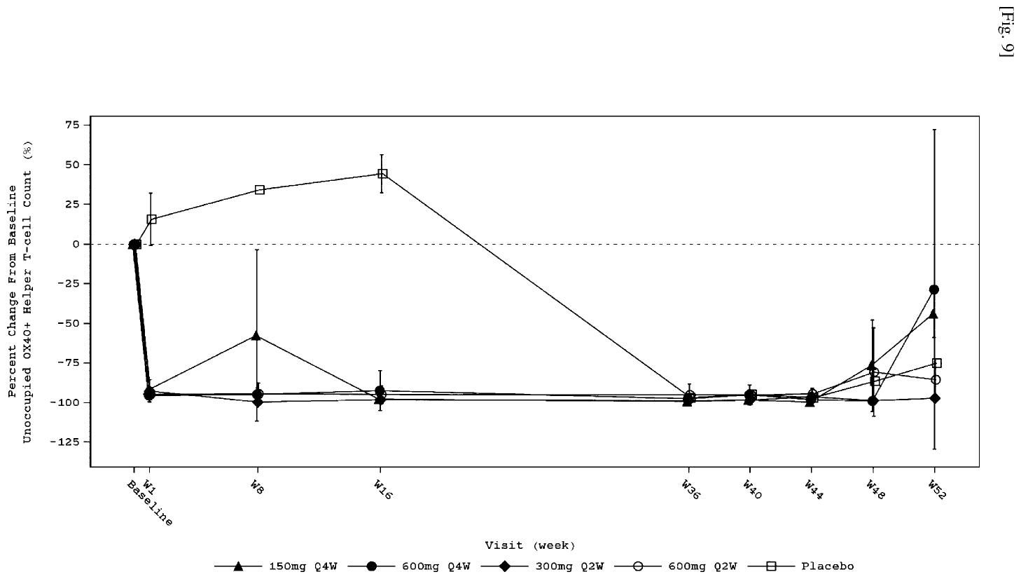

The percentage changes from baseline in the total OX40-positive helper T-cell counts (%) in blood of the administration groups are shown in FIG. 8. Further, the percentage changes from baseline in counts of cells (unoccupied OX40-positive cells) to which KHK4083 is not bound (%) among the OX40-positive helper T-cells in blood of the administration groups are shown in FIG. 9. The error bar shows SD. The results shown in FIG. 8 and FIG. 9 demonstrated that the blood OX40-positive helper T-cell counts decreased after the administration of KHK4083 in all the KHK4083 administration groups, and that KHK4083 is bound to OX40 on the remaining OX40-positive helper T-cells. Moreover, this effect lasted at least until Week 52 after the final administration at Week 32 or 34. The decrease in the blood OX40-positive helper T-cells by KHK4083 over a long period of time, and the lasting inhibition by KHK4083 by lasting binding to OX40 on the remaining OX40-positive cells are believed to contribute to the lasting efficacy after completion of administration.

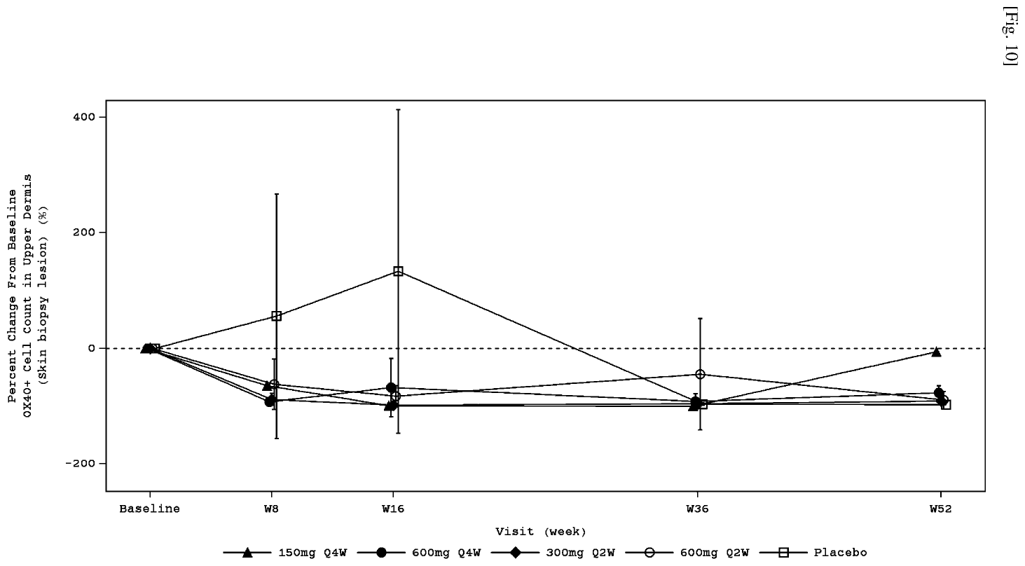

The OX40-positive cell count in the upper dermis was analyzed by immunohistochemical staining. The percentage changes from baseline in the OX40-positive cell counts (%) of the administration groups are shown in FIG. 10. The error bar shows SD. From this figure, it was found that the OX40-positive cell counts in the upper dermis decreased after the administration of KHK4083 in all the KHK4083 administration groups. The decrease in the cell counts lasted at least until Week 52 after the final administration at Week 32 or 34. As a result of suppressing the blood OX40-positive helper T-cells as described above, the OX40-positive cell count in the skin tissues also decreased and the inflammatory reaction in the topical skin is believed to improve, and this is also believed to contribute to the lasting efficacy of KHK4083.

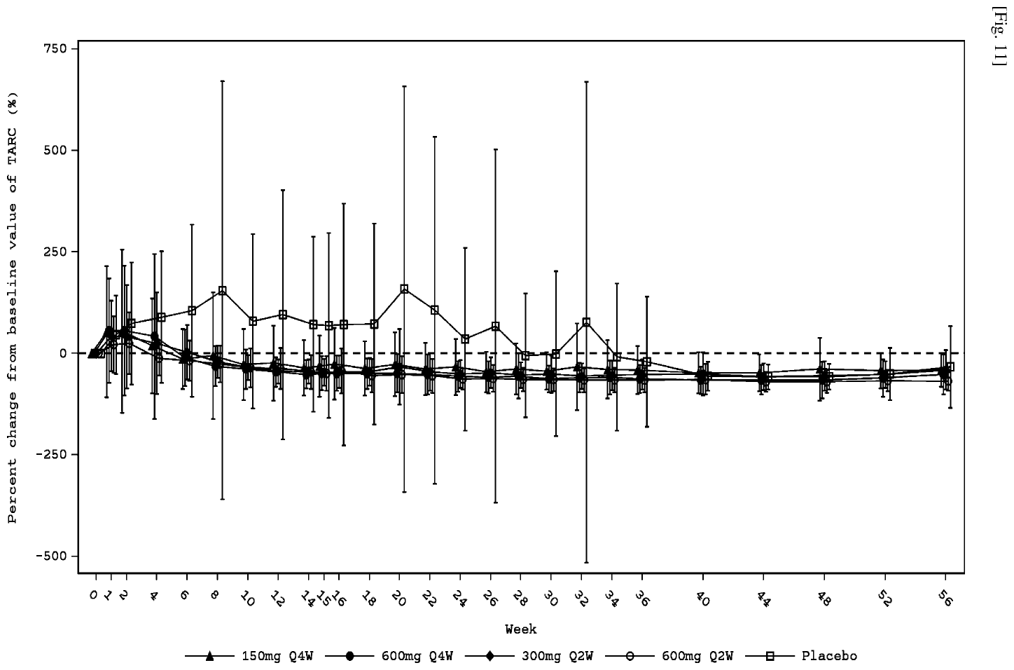

The percentage changes from baseline in the TARC value in blood (%) of the administration groups are shown in FIG. 11. The error bar shows SD. From this figure, it was found that the TARC value in blood decreases from baseline compared to the placebo group, and the decrease is maintained until Week 56 in all the KHK4083 administration groups. TARC is a ligand for CCR4 expressed in helper T cells mainly called Th2 and involved mainly in diseases such as atopic dermatitis and asthma, and is a type of chemokine that allows Th2 to migrate to an inflammation site. TARC is known to be a sensitive disease marker for atopic dermatitis, and it is believed that KHK4083 continuously exhibits its efficacy also from the viewpoint of pathological molecular mechanism of atopic dermatitis.

### Descriptive Summary of EASI Score at Each Scheduled Visit at Week 1, 2, and 4:

TABLE 9
Descriptive summary of EASI Score at each scheduled visit
(Full Analysis Set)

| | | | | | | | Placebo/ |
|---|---|---|---|---|---|---|---|
| | | | KHK4083 | KHK4083 | KHK4083 | KHK4083 | KHK4083 |
| | | | 150 mg | 600 mg | 300 mg | 600 mg | 600 mg |
| | | | Q4W | Q4W | Q2W | Q2W | Q2W |
| Item | Visit | Statistics | N = 52 | N = 52 | N = 52 | N = 54 | N = 57 |
| Percent Changes | Week 1 | n | 50 | 51 | 50 | 54 | 54 |
| from Baseline | | Mean | −2.5 | −9.5 | −8.5 | −7.0 | −2.9 |
| | | SD | 22.3 | 20.6 | 34.5 | 28.8 | 29.4 |
| | | Min | | | | | |
| | | Median | | | | | |
| | | Max | | | | | |
| | Week 2 | n | 46 | 50 | 48 | 53 | 50 |
| | | Mean | −6.7 | −14.3 | −18.4 | −15.6 | −2.6 |
| | | SD | 29.7 | 25.8 | 38.1 | 34.0 | 38.9 |
| | | Min | | | | | |
| | | Median | | | | | |
| | | Max | | | | | |
| | Week 4 | n | 46 | 49 | 46 | 51 | 44 |
| | | Mean | −19.6 | −19.4 | −26.4 | −23.4 | −8.5 |
| | | SD | 39.8 | 28.3 | 46.2 | 33.8 | 26.8 |
| | | Min | | | | | |
| | | Median | | | | | |
| | | Max | | | | | |

EASI = Eczema Area and Severity Index,
Max = maximum,
Min = minimum,
SD = standard deviation.
Note:
Subjects in Placebo group were switched to KHK4083 600 mg Q2W after Treatment A Period.

### Descriptive Summary of EASI Score at Each Scheduled Visit at Week 6, 8, and 10:

TABLE 10
Descriptive summary of EASI Score at each scheduled visit
(Full Analysis Set)

| | | | | | | | Placebo/ |
|---|---|---|---|---|---|---|---|
| | | | KHK4083 | KHK4083 | KHK4083 | KHK4083 | KHK4083 |
| | | | 150 mg | 600 mg | 300 mg | 600 mg | 600 mg |
| | | | Q4W | Q4W | Q2W | Q2W | Q2W |
| Item | Visit | Statistics | N = 52 | N = 52 | N = 52 | N = 54 | N = 57 |
| Percent Changes | Week 6 | n | 42 | 48 | 42 | 51 | 41 |
| from Baseline | | Mean | −28.8 | −32.5 | −43.4 | −35.8 | −11.1 |
| | | SD | 37.6 | 34.6 | 42.0 | 35.1 | 35.6 |
| | | Min | | | | | |
| | | Median | | | | | |
| | | Max | | | | | |
| | Week 8 | n | 42 | 45 | 42 | 50 | 40 |
| | | Mean | −40.3 | −45.5 | −52.2 | −42.1 | −19.2 |
| | | SD | 43.6 | 29.7 | 34.9 | 35.6 | 42.7 |
| | | Min | | | | | |
| | | Median | | | | | |
| | | Max | | | | | |
| | Week 10 | n | 43 | 44 | 43 | 48 | 39 |
| | | Mean | −51.5 | −51.9 | −60.4 | −48.2 | −21.1 |
| | | SD | 35.4 | 33.8 | 33.0 | 34.5 | 38.4 |
| | | Min | | | | | |
| | | Median | | | | | |
| | | Max | | | | | |

EASI = Eczema Area and Severity Index,
Max = maximum,
Min = minimum,
SD = standard deviation.
Note:
Subjects in Placebo group were switched to KHK4083 600 mg Q2W after Treatment A Period.

### Descriptive Summary of EASI Score at Each Scheduled Visit at Week 12, 14, and 15:

TABLE 11
Descriptive summary of EASI Score at each scheduled visit
(Full Analysis Set)

| | | | | | | | Placebo/ |
|---|---|---|---|---|---|---|---|
| | | | KHK4083 | KHK4083 | KHK4083 | KHK4083 | KHK4083 |
| | | | 150 mg | 600 mg | 300 mg | 600 mg | 600 mg |
| | | | Q4W | Q4W | Q2W | Q2W | Q2W |
| Item | Visit | Statistics | N = 52 | N = 52 | N = 52 | N = 54 | N = 57 |
| Percent Changes | Week 12 | n | 43 | 45 | 42 | 48 | 36 |
| from Baseline | | Mean | −56.7 | −58.4 | −68.3 | −50.9 | −29.5 |
| | | SD | 33.8 | 26.5 | 28.1 | 31.9 | 39.7 |
| | | Min | | | | | |
| | | Median | | | | | |
| | | Max | | | | | |
| | Week 14 | n | 42 | 42 | 42 | 48 | 34 |
| | | Mean | −62.5 | −62.1 | −71.1 | −61.5 | −32.1 |
| | | SD | 29.2 | 26.9 | 28.9 | 25.9 | 38.1 |
| | | Min | | | | | |
| | | Median | | | | | |
| | | Max | | | | | |
| | Week 15 | n | 40 | 43 | 41 | 48 | 37 |
| | | Mean | −67.1 | −62.2 | −74.1 | −62.2 | −35.3 |
| | | SD | 29.4 | 28.5 | 24.9 | 27.3 | 41.4 |
| | | Min | | | | | |
| | | Median | | | | | |
| | | Max | | | | | |

EASI = Eczema Area and Severity Index,
Max = maximum,
Min = minimum,
SD = standard deviation.
Note:
Subjects in Placebo group were switched to KHK4083 600 mg Q2W after Treatment A Period.

### Descriptive Summary of EASI Score at Each Scheduled Visit at Week 16, 18, and 20:

TABLE 12
Descriptive summary of EASI Score at each scheduled visit
(Full Analysis Set)

| | | | | | | | Placebo/ |
|---|---|---|---|---|---|---|---|
| | | | KHK4083 | KHK4083 | KHK4083 | KHK4083 | KHK4083 |
| | | | 150 mg | 600 mg | 300 mg | 600 mg | 600 mg |
| | | | Q4W | Q4W | Q2W | Q2W | Q2W |
| Item | Visit | Statistics | N = 52 | N = 52 | N = 52 | N = 54 | N = 57 |
| Percent Changes | Week 16 | n | 41 | 44 | 43 | 47 | 36 |
| from Baseline | | Mean | −67.0 | −63.2 | −77.2 | −63.6 | −37.4 |
| | | SD | 32.1 | 29.7 | 22.9 | 30.9 | 42.4 |
| | | Min | | | | | |
| | | Median | | | | | |
| | | Max | | | | | |
| | Week 18 | n | 41 | 43 | 40 | 46 | 33 |
| | | Mean | −67.1 | −66.1 | −75.5 | −63.9 | −32.6 |
| | | SD | 30.8 | 27.6 | 23.3 | 52.3 | 46.6 |
| | | Min | | | | | |
| | | Median | | | | | |
| | | Max | | | | | |
| | Week 20 | n | 41 | 41 | 39 | 45 | 30 |
| | | Mean | −69.7 | −70.6 | −79.0 | −74.4 | −37.9 |
| | | SD | 31.1 | 26.8 | 21.0 | 27.0 | 50.8 |
| | | Min | | | | | |
| | | Median | | | | | |
| | | Max | | | | | |

EASI = Eczema Area and Severity Index,
Max = maximum,
Min = minimum,
SD = standard deviation.
Note:
Subjects in Placebo group were switched to KHK4083 600 mg Q2W after Treatment A Period.

### Descriptive Summary of EASI Score at Each Scheduled Visit at Week 22, 24, and 26:

TABLE 13
Descriptive summary of EASI Score at each scheduled visit
(Full Analysis Set)

| | | | | | | | Placebo/ |
|---|---|---|---|---|---|---|---|
| | | | KHK4083 | KHK4083 | KHK4083 | KHK4083 | KHK4083 |
| | | | 150 mg | 600 mg | 300 mg | 600 mg | 600 mg |
| | | | Q4W | Q4W | Q2W | Q2W | Q2W |
| Item | Visit | Statistics | N = 52 | N = 52 | N = 52 | N = 54 | N = 57 |
| Percent Changes | Week 22 | n | 39 | 40 | 41 | 44 | 31 |
| from Baseline | | Mean | −74.3 | −75.6 | −80.8 | −79.4 | −49.3 |
| | | SD | 29.7 | 26.1 | 22.4 | 23.7 | 44.4 |
| | | Min | | | | | |
| | | Median | | | | | |
| | | Max | | | | | |
| | Week 24 | n | 40 | 40 | 41 | 42 | 30 |
| | | Mean | −71.7 | −75.5 | −85.3 | −80.4 | −49.7 |
| | | SD | 32.0 | 25.9 | 22.0 | 22.5 | 46.1 |
| | | Min | | | | | |
| | | Median | | | | | |
| | | Max | | | | | |
| | Week 26 | n | 38 | 38 | 39 | 43 | 26 |
| | | Mean | −75.3 | −78.2 | −86.3 | −81.0 | −70.1 |
| | | SD | 27.8 | 23.3 | 20.8 | 25.0 | 29.5 |
| | | Min | | | | | |
| | | Median | | | | | |
| | | Max | | | | | |

EASI = Eczema Area and Severity Index,
Max = maximum,
Min = minimum,
SD = standard deviation.
Note:
Subjects in Placebo group were switched to KHK4083 600 mg Q2W after Treatment A Period.

### Descriptive Summary of EASI Score at Each Scheduled Visit at Week 28, 30, and 32:

TABLE 14
Descriptive summary of EASI Score at each scheduled visit
(Full Analysis Set)

| | | | | | | | Placebo/ |
|---|---|---|---|---|---|---|---|
| | | | KHK4083 | KHK4083 | KHK4083 | KHK4083 | KHK4083 |
| | | | 150 mg | 600 mg | 300 mg | 600 mg | 600 mg |
| | | | Q4W | Q4W | Q2W | Q2W | Q2W |
| Item | Visit | Statistics | N = 52 | N = 52 | N = 52 | N = 54 | N = 57 |
| Percent Changes | Week 28 | n | 36 | 38 | 38 | 40 | 26 |
| from Baseline | | Mean | −78.7 | −77.5 | −84.8 | −83.9 | −70.6 |
| | | SD | 26.1 | 22.1 | 26.3 | 19.6 | 27.5 |
| | | Min | | | | | |
| | | Median | | | | | |
| | | Max | | | | | |
| | Week 30 | n | 34 | 37 | 38 | 40 | 25 |
| | | Mean | −80.8 | −81.1 | −87.3 | −86.5 | −74.5 |
| | | SD | 22.5 | 19.0 | 21.1 | 17.0 | 25.6 |
| | | Min | | | | | |
| | | Median | | | | | |
| | | Max | | | | | |
| | Week 32 | n | 33 | 38 | 37 | 40 | 23 |
| | | Mean | −82.5 | −82.7 | −90.5 | −87.9 | −82.4 |
| | | SD | 23.9 | 17.6 | 13.4 | 14.4 | 17.4 |
| | | Min | | | | | |
| | | Median | | | | | |
| | | Max | | | | | |

EASI = Eczema Area and Severity Index,
Max = maximum,
Min = minimum,
SD = standard deviation.
Note:
Subjects in Placebo group were switched to KHK4083 600 mg Q2W after Treatment A Period.

### Descriptive Summary of EASI Score at Each Scheduled Visit at Week 34, 36, and 40:

TABLE 15
Descriptive summary of EASI Score at each scheduled visit
(Full Analysis Set)

| | | | | | | | Placebo/ |
|---|---|---|---|---|---|---|---|
| | | | KHK4083 | KHK4083 | KHK4083 | KHK4083 | KHK4083 |
| | | | 150 mg | 600 mg | 300 mg | 600 mg | 600 mg |
| | | | Q4W | Q4W | Q2W | Q2W | Q2W |
| Item | Visit | Statistics | N = 52 | N = 52 | N = 52 | N = 54 | N = 57 |
| Percent Changes | Week 34 | n | 33 | 38 | 35 | 37 | 23 |
| from Baseline | | Mean | −83.6 | −80.5 | −91.3 | −87.1 | −81.8 |
| | | SD | 23.6 | 19.4 | 12.6 | 20.0 | 22.3 |
| | | Min | | | | | |
| | | Median | | | | | |
| | | Max | | | | | |
| | Week 36 | n | 34 | 37 | 36 | 37 | 25 |
| | | Mean | −84.5 | −83.5 | −93.0 | −87.2 | −82.2 |
| | | SD | 23.1 | 20.1 | 10.0 | 14.3 | 23.2 |
| | | Min | | | | | |
| | | Median | | | | | |
| | | Max | | | | | |
| | Week 40 | n | 26 | 33 | 30 | 30 | 21 |
| | | Mean | −88.0 | −86.5 | −91.7 | −88.4 | −83.4 |
| | | SD | 16.4 | 14.7 | 14.0 | 12.4 | 26.2 |
| | | Min | | | | | |
| | | Median | | | | | |
| | | Max | | | | | |

EASI = Eczema Area and Severity Index,
Max = maximum,
Min = minimum,
SD = standard deviation.
Note:
Subjects in Placebo group were switched to KHK4083 600 mg Q2W after Treatment A Period.

### Descriptive Summary of EASI Score at Each Scheduled Visit at Week 44, 48, and 52:

TABLE 16
Descriptive summary of EASI Score at each scheduled visit
(Full Analysis Set)

| | | | | | | | Placebo/ |
|---|---|---|---|---|---|---|---|
| | | | KHK4083 | KHK4083 | KHK4083 | KHK4083 | KHK4083 |
| | | | 150 mg | 600 mg | 300 mg | 600 mg | 600 mg |
| | | | Q4W | Q4W | Q2W | Q2W | Q2W |
| Item | Visit | Statistics | N = 52 | N = 52 | N = 52 | N = 54 | N = 57 |
| Percent | Week 44 | n | 24 | 32 | 27 | 29 | 17 |
| Changes | | Mean | −89.1 | −80.6 | −94.4 | −88.1 | −88.8 |
| from Baseline | | SD | 18.1 | 39.7 | 6.1 | 15.9 | 23.7 |
| | | Min | | | | | |
| | | Median | | | | | |
| | | Max | | | | | |
| | Week 48 | n | 19 | 28 | 24 | 25 | 14 |
| | | Mean | −83.2 | −90.6 | −94.6 | −91.2 | −86.7 |
| | | SD | 25.8 | 11.7 | 6.7 | 12.3 | 29.3 |
| | | Min | | | | | |
| | | Median | | | | | |
| | | Max | | | | | |
| | Week 52 | n | 15 | 29 | 17 | 21 | 14 |
| | | Mean | −89.9 | −91.3 | −93.7 | −91.8 | −92.2 |
| | | SD | 15.4 | 13.0 | 9.8 | 11.5 | 11.1 |
| | | Min | | | | | |
| | | Median | | | | | |
| | | Max | | | | | |

EASI = Eczema Area and Severity Index,
Max = maximum,
Min = minimum,
SD = standard deviation.
Note:
Subjects in Placebo group were switched to KHK4083 600 mg Q2W after Treatment A Period.

### Descriptive Summary of EASI Score at Each Scheduled Visit at Week 56:

TABLE 17
Descriptive summary of EASI Score at each scheduled visit (Full Analysis Set)

| | | | | | | | Placebo/ |
|---|---|---|---|---|---|---|---|
| | | | KHK4083 | KHK4083 | KHK4083 | KHK4083 | KHK4083 |
| | | | 150 mg | 600 mg | 300 mg | 600 mg | 600 mg |
| | | | Q4W | Q4W | Q2W | Q2W | Q2W |
| Item | Visit | Statistics | N = 52 | N = 52 | N = 52 | N = 54 | N = 57 |
| Percent Changes | Week 56 | n | 14 | 24 | 12 | 16 | 11 |
| from Baseline | | mean | −84.3 | −87.0 | −93.3 | −89.4 | −91.4 |
| | | SD | 21.0 | 22.5 | 10.7 | 14.5 | 12.6 |
| | | Min | | | | | |
| | | Median | | | | | |
| | | Max | | | | | |

EASI = Eczema Area and Severity Index,
Max = maximum,
Min = minimum,
SD = standard deviation.
Note:
Subjects in Placebo group were switched to KHK4083 600 mg Q2W after Treatment A Period.

### Number of Patients and Number of Patients Who Achieved EASI-75 of Administration Groups:

TABLE 18
Variable: EASI-75

| | Number of Subjects with assessment | | | | | Number and (%) of Subjects Achieved | | | | | | | | | |
|---|---|---|---|---|---|---|---|---|---|---|---|---|---|---|---|
| | | | | | Placebo/ | KHK4083 | | KHK4083 | | KHK4083 | | KHK4083 | | Placebo | |
| | KHK4083 | KHK4083 | KHK4083 | KHK4083 | KHK4083 | 150 mg | | 600 mg | | 300 mg | | 600 mg | | KHK4083 | |
| | 150 mg | 600 mg | 300 mg | 600 mg | 600 mg | Q4W | | Q4W | | Q2W | | Q2W | | 600 mg Q2W | |
| Visit | Q4W | Q4W | Q2W | Q2W | Q2W | N = 52 | | N = 52 | | N = 52 | | N = 54 | | N = 57 | |
| Week | 42 | 48 | 42 | 51 | 41 | 5 | (9.6) | 5 | (9.6) | 12 | (23.1) | 8 | (14.8) | 2 | (3.5) |
| Week 8 | 42 | 45 | 42 | 50 | 40 | 11 | (21.2) | 7 | (13.5) | 10 | (19.2) | 11 | (20.4) | 3 | (5.3) |
| Week 10 | 43 | 44 | 43 | 48 | 39 | 11 | (21.2) | 11 | (21.2) | 17 | (32.7) | 13 | (24.1) | 5 | (8.8) |
| Week 12 | 43 | 45 | 42 | 48 | 36 | 13 | (25.0) | 14 | (26.9) | 24 | (46.2) | 16 | (29.6) | 4 | (7.0) |
| Week 14 | 42 | 42 | 42 | 48 | 34 | 17 | (32.7) | 17 | (32.7) | 25 | (48.1) | 17 | (31.5) | 5 | (8.8) |
| Week 15 | 40 | 43 | 41 | 48 | 37 | 19 | (36.5) | 18 | (34.6) | 25 | (48.1) | 18 | (33.3) | 7 | (12.3) |
| Week 16 | 41 | 44 | 43 | 47 | 36 | 23 | (44.2) | 21 | (40.4) | 28 | (53.8) | 21 | (38.9) | 6 | (10.5) |
| Week 18 | 41 | 43 | 40 | 46 | 33 | 23 | (42.3) | 21 | (40.4) | 23 | (44.2) | 23 | (42.6) | 5 | (8.8) |
| Week 20 | 41 | 41 | 39 | 45 | 30 | 24 | (46.2) | 21 | (40.4) | 26 | (50.0) | 27 | (50.0) | 8 | (14.0) |
| Week 22 | 39 | 40 | 41 | 44 | 31 | 27 | (51.9) | 29 | (55.8) | 30 | (57.7) | 30 | (55.6) | 11 | (19.3) |

### Number of Patients and Number of Patients Who Achieved EASI-75 of Administration Groups:

TABLE 19
Variable: EASI-75

| | Number of Subjects with assessment | | | | | Number and (%) of Subjects Achieved | | | | | | | | | |
|---|---|---|---|---|---|---|---|---|---|---|---|---|---|---|---|
| | | | | | Placebo/ | KHK4083 | | KHK4083 | | KHK4083 | | KHK4083 | | Placebo/ | |
| | KHK4083 | KHK4083 | KHK4083 | KHK4083 | KHK4083 | 150 mg | | 600 mg | | 300 mg | | 600 mg | | KHK4083 | |
| | 150 mg | 600 mg | 300 mg | 600 mg | 600 mg | Q4W | | Q4W | | Q2W | | Q2W | | 600 mg Q2W | |
| Visit | Q4W | Q4W | Q2W | Q2W | Q2W | N = 52 | | N = 52 | | N = 52 | | N = 54 | | N = 57 | |
| Week 24 | 40 | 40 | 41 | 42 | 30 | 25 | (48.1) | 28 | (53.8) | 34 | (65.4) | 29 | (53.7) | 11 | (19.3) |
| Week 26 | 38 | 38 | 39 | 43 | 26 | 25 | (48.1) | 29 | (55.8) | 34 | (65.4) | 33 | (61.1) | 17 | (29.8) |
| Week 28 | 36 | 38 | 38 | 40 | 26 | 26 | (50.0) | 26 | (50.0) | 33 | (63.5) | 32 | (59.3) | 17 | (29.8) |
| Week 30 | 34 | 37 | 38 | 40 | 25 | 24 | (46.2) | 26 | (50.0) | 32 | (61.5) | 32 | (59.3) | 18 | (31.6) |
| Week 32 | 33 | 38 | 37 | 40 | 23 | 23 | (44.2) | 29 | (55.8) | 31 | (59.6) | 35 | (64.8) | 19 | (33.3) |
| Week 34 | 33 | 38 | 35 | 37 | 23 | 23 | (44.2) | 27 | (51.9) | 30 | (57.7) | 31 | (57.4) | 19 | (33.3) |
| Week 36 | 34 | 37 | 36 | 37 | 25 | 27 | (51.9) | 30 | (57.7) | 33 | (63.5) | 31 | (57.4) | 20 | (35.1) |
| Week 40 | 26 | 33 | 30 | 30 | 21 | 23 | (44.2) | 27 | (51.9) | 27 | (51.9) | 26 | (48.1) | 17 | (29.8) |
| Week 44 | 24 | 32 | 27 | 29 | 17 | 21 | (40.4) | 25 | (48.1) | 27 | (51.9) | 24 | (44.4) | 16 | (28.1) |
| Week 48 | 19 | 28 | 24 | 25 | 14 | 16 | (30.8) | 26 | (50.0) | 24 | (46.2) | 23 | (42.6) | 13 | (22.8) |
| Week 52 | 15 | 29 | 17 | 21 | 14 | 13 | (25.0) | 26 | (50.0) | 16 | (30.8) | 19 | (35.2) | 12 | (21.1) |
| Week 56 | 14 | 24 | 12 | 16 | 11 | 10 | (19.2) | 20 | (38.5) | 11 | (21.2) | 13 | (24.1) | 10 | (17.5) |

### Number of Patients and Number of Patients Who Achieved EASI-90 of Administration Groups:

TABLE 20
Variable: EASI-90

| | Number of Subjects with assessment | | | | | Number and (%) of Subjects Achieved | | | | | | | | | |
|---|---|---|---|---|---|---|---|---|---|---|---|---|---|---|---|
| | | | | | Placebo/ | KHK4083 | | KHK4083 | | KHK4083 | | KHK4083 | | Placebo/ | |
| | KHK4083 | KHK4083 | KHK4083 | KHK4083 | KHK4083 | 150 mg | | 600 mg | | 300 mg | | 600 mg | | KHK4083 | |
| | 150 mg | 600 mg | 300 mg | 600 mg | 600 mg | Q4W | | Q4W | | Q2W | | Q2W | | 600 mg Q2W | |
| Visit | Q4W | Q4W | Q2W | Q2W | Q2W | N = 52 | | N = 52 | | N = 52 | | N = 54 | | N = 57 | |
| Week 10 | 43 | 44 | 43 | 48 | 39 | 7 | (13.5) | 3 | (5.8) | 8 | (15.4) | 5 | (9.3) | 1 | (1.8) |
| Week 12 | 43 | 45 | 42 | 48 | 36 | 9 | (17.3) | 4 | (7.7) | 10 | (19.2) | 5 | (9.3) | 2 | (3.5) |
| Week 14 | 42 | 42 | 42 | 48 | 34 | 6 | (11.5) | 6 | (11.5) | 16 | (30.8) | 8 | (14.8) | 2 | (3.5) |
| Week 15 | 40 | 43 | 41 | 48 | 37 | 11 | (21.2) | 6 | (11.5) | 17 | (32.7) | 8 | (14.8) | 3 | (5.3) |
| Week 16 | 41 | 44 | 43 | 47 | 36 | 10 | (19.2) | 6 | (11.5) | 19 | (36.5) | 10 | (18.5) | 2 | (3.5) |
| Week 18 | 41 | 43 | 40 | 46 | 33 | 9 | (17.3) | 11 | (21.2) | 15 | (28.8) | 13 | (24.1) | 3 | (5.3) |
| Week 20 | 41 | 41 | 39 | 45 | 30 | 14 | (26.9) | 14 | (26.9) | 17 | (32.7) | 14 | (25.9) | 3 | (5.3) |
| Week 22 | 39 | 40 | 41 | 44 | 31 | 16 | (30.8) | 15 | (28.8) | 20 | (38.5) | 17 | (31.5) | 5 | (8.8) |

### Proportion of Achievement for EASI-50 (Each Week):

TABLE 21
Variable: EASI-50

| | Number of Subjects with assessment | | | | | Number and (%) of Subjects Achieved | | | | | | | | | |
|---|---|---|---|---|---|---|---|---|---|---|---|---|---|---|---|
| | | | | | Placebo/ | KHK4083 | | KHK4083 | | KHK4083 | | KHK4083 | | Placebo/ | |
| | KHK4083 | KHK4083 | KHK4083 | KHK4083 | KHK4083 | 150 mg | | 600 mg | | 300 mg | | 600 mg | | KHK4083 | |
| | 150 mg | 600 mg | 300 mg | 600 mg | 600 mg | Q4W | | Q4W | | Q2W | | Q2W | | 600 mg Q2W | |
| Visit | Q4W | Q4W | Q2W | Q2W | Q2W | N = 52 | | N = 52 | | N = 52 | | N = 54 | | N = 57 | |
| Week 1 | 50 | 51 | 50 | 54 | 54 | 1 | (1.9) | 2 | (3.8) | 7 | (13.5) | 3 | (5.6) | 1 | (1.8) |
| Week 2 | 46 | 50 | 48 | 53 | 50 | 5 | (9.6) | 7 | (13.5) | 11 | (21.2) | 8 | (14.8) | 3 | (5.3) |
| Week 4 | 46 | 49 | 46 | 52 | 44 | 10 | (19.2) | 9 | (17.3) | 14 | (26.9) | 11 | (20.4) | 2 | (3.5) |
| Week 6 | 42 | 48 | 42 | 51 | 41 | 12 | (23.1) | 17 | (32.7) | 22 | (42.3) | 20 | (37.0) | 5 | (8.8) |
| Week 8 | 42 | 45 | 42 | 50 | 40 | 18 | (34.6) | 21 | (40.4) | 29 | (55.8) | 24 | (44.4) | 10 | (17.5) |
| Week 10 | 43 | 44 | 43 | 48 | 39 | 26 | (50.0) | 29 | (55.8) | 32 | (61.5) | 26 | (48.1) | 10 | (17.5) |
| Week 12 | 43 | 45 | 42 | 48 | 36 | 28 | (53.8) | 31 | (59.6) | 34 | (65.4) | 27 | (50.0) | 13 | (22.8) |
| Week 14 | 42 | 42 | 42 | 48 | 34 | 31 | (59.6) | 31 | (59.6) | 33 | (63.5) | 34 | (63.0) | 12 | (21.1) |
| Week 15 | 40 | 43 | 41 | 48 | 37 | 30 | (57.7) | 30 | (57.7) | 33 | (63.5) | 31 | (57.4) | 15 | (26.3) |
| Weak 16 | 41 | 44 | 43 | 47 | 36 | 30 | (57.7) | 31 | (59.6) | 36 | (69.2) | 35 | (64.8) | 17 | (29.8) |
| Week 18 | 41 | 43 | 40 | 46 | 33 | 32 | (61.5) | 32 | (61.5) | 34 | (65.4) | 36 | (66.7) | 16 | (28.1) |
| Week 20 | 41 | 41 | 39 | 45 | 30 | 33 | (63.5) | 32 | (61.5) | 36 | (69.2) | 39 | (72.2) | 17 | (29.8) |
| Week 22 | 39 | 40 | 41 | 44 | 31 | 32 | (61.5) | 34 | (65.4) | 36 | (69.2) | 41 | (75.9) | 19 | (33.3) |

### Proportion of Achievement for EASI-50 (Each Week):

TABLE 22
Variable: EASI-50

| | Number of Subjects with assessment | | | | | Number and (%) of Subjects Achieved | | | | | | | | | |
|---|---|---|---|---|---|---|---|---|---|---|---|---|---|---|---|
| | | | | | Placebo/ | KHK 4083 | | KHK4083 | | KHK4083 | | KHK4083 | | Placebo/ | |
| | KHK4083 | KHK4083 | KHK4083 | KHK4083 | KHK4083 | 150 mg | | 600 mg | | 300 mg | | 600 mg | | KHK4083 | |
| | 150 mg | 600 mg | 300 mg | 600 mg | 600 mg | Q4W | | Q4W | | Q2W | | Q2W | | 600 mg Q2W | |
| Visit | Q4W | Q4W | Q2W | Q2W | Q2W | N = 52 | | N = 52 | | N = 52 | | N = 54 | | N = 57 | |
| Week 24 | 40 | 40 | 41 | 42 | 30 | 32 | (61.5) | 34 | (65.4) | 39 | (75.0) | 40 | (74.1) | 19 | (33.3) |
| Week 26 | 38 | 38 | 39 | 43 | 26 | 31 | (59.6) | 31 | (59.6) | 35 | (67.3) | 39 | (72.2) | 21 | (36.8) |
| Week 28 | 36 | 38 | 38 | 40 | 26 | 30 | (57.7) | 33 | (63.5) | 34 | (65.4) | 38 | (70.4) | 21 | (36.8) |
| Week 30 | 34 | 37 | 38 | 40 | 25 | 30 | (57.7) | 35 | (67.3) | 36 | (69.2) | 39 | (72.2) | 21 | (36.8) |
| Week 32 | 33 | 38 | 37 | 40 | 23 | 30 | (57.7) | 35 | (67.3) | 37 | (71.2) | 38 | (70.4) | 21 | (36.8) |
| Week 34 | 33 | 38 | 35 | 37 | 23 | 31 | (59.6) | 36 | (69.2) | 35 | (67.3) | 35 | (64.8) | 20 | (35.1) |
| Week 36 | 34 | 37 | 36 | 37 | 25 | 31 | (59.6) | 34 | (65.4) | 36 | (69.2) | 36 | (66.7) | 23 | (40.4) |
| Week 40 | 26 | 34 | 30 | 30 | 22 | 24 | (46.2) | 34 | (65.4) | 29 | (55.8) | 30 | (55.6) | 20 | (35.1) |
| Week 44 | 24 | 34 | 29 | 30 | 19 | 23 | (44.2) | 31 | (59.6) | 29 | (55.8) | 29 | (53.7) | 18 | (31.6) |
| Week 48 | 22 | 32 | 28 | 26 | 17 | 19 | (36.5) | 32 | (61.5) | 28 | (53.8) | 26 | (48.1) | 16 | (28.1) |
| Week 52 | 18 | 33 | 23 | 24 | 17 | 17 | (32.7) | 33 | (63.5) | 23 | (44.2) | 24 | (44.4) | 17 | (29.8) |
| Week 56 | 19 | 28 | 18 | 20 | 16 | 18 | (34.6) | 25 | (48.1) | 17 | (32.7) | 20 | (37.0) | 16 | (28.1) |

### Proportion of Achievement for EASI-75 (Each Week):

TABLE 23
Variable: EASI-75

| | Number of Subjects with assessment | | | | | Number and (%) of Subjects Achieved | | | | | | | | | |
|---|---|---|---|---|---|---|---|---|---|---|---|---|---|---|---|
| | | | | | Placebo/ | KHK4083 | | KHK4083 | | KHK4083 | | KHK4083 | | Placebo/ | |
| | KHK4083 | KHK4083 | KHK4083 | KHK4083 | KHK4083 | 150 mg | | 600 mg | | 300 mg | | 600 mg | | KHK4083 | |
| | 150 mg | 600 mg | 300 mg | 600 mg | 600 mg | Q4W | | Q4H | | Q2W | | Q2W | | 600 mg Q2W | |
| Visit | Q4H | Q4W | Q2W | Q2W | Q2W | N = 52 | | N = 52 | | N = 52 | | N = 54 | | N = 57 | |
| Week 1 | 50 | 51 | 50 | 54 | 54 | 0 | (0.0) | 0 | (0.0) | 0 | (0.0) | 1 | (1.9) | 0 | (0.0) |
| Week 2 | 46 | 50 | 48 | 53 | 50 | 0 | (0.0) | 1 | (1.9) | 3 | (5.8) | 3 | (5.6) | 0 | (0.0) |
| Week 4 | 46 | 49 | 46 | 52 | 44 | 6 | (11.5) | 0 | (0.0) | 6 | (11.5) | 7 | (13.0) | 1 | (1.8) |
| Week 6 | 42 | 48 | 42 | 51 | 41 | 5 | (9.6) | 5 | (9.6) | 12 | (23.1) | 8 | (14.8) | 2 | (3.5) |
| Week 8 | 42 | 45 | 42 | 50 | 40 | 11 | (21.2) | 7 | (13.5) | 10 | (19.2) | 11 | (20.4) | 3 | (5.3) |
| Week 10 | 43 | 44 | 43 | 48 | 39 | 11 | (21.2) | 11 | (21.2) | 17 | (32.7) | 13 | (24.1) | 5 | (8.8) |
| Week 12 | 43 | 45 | 42 | 48 | 36 | 13 | (25.0) | 14 | (26.9) | 24 | (46.2) | 16 | (29.6) | 4 | (7.0) |
| Week 14 | 42 | 42 | 42 | 48 | 34 | 17 | (32.7) | 17 | (32.7) | 25 | (48.1) | 17 | (31.5) | 5 | (8.8) |
| Week 15 | 40 | 43 | 41 | 48 | 37 | 19 | (36.5) | 18 | (34.6) | 25 | (48.1) | 18 | (33.3) | 7 | (12.3) |
| Week 16 | 41 | 44 | 43 | 47 | 36 | 23 | (44.2) | 21 | (40.4) | 28 | (53.8) | 21 | (38.9) | 6 | (10.5) |
| Week 18 | 41 | 43 | 40 | 46 | 33 | 22 | (42.3) | 21 | (40.4) | 23 | (44.2) | 23 | (42.6) | 5 | (8.8) |
| Week 20 | 41 | 41 | 39 | 45 | 30 | 24 | (46.2) | 21 | (40.4) | 26 | (50.0) | 27 | (50.0) | 8 | (14.0) |
| Week 22 | 39 | 40 | 41 | 44 | 31 | 27 | (51.9) | 29 | (55.8) | 30 | (57.7) | 30 | (55.6) | 11 | (19.3) |

### Proportion of Achievement for EASI-75 (Each Week):

TABLE 24
Variable: EASI-75

| | Number of Subjects with assessment | | | | | Number and (%) of Subjects Achieved | | | | | | | | | |
|---|---|---|---|---|---|---|---|---|---|---|---|---|---|---|---|
| | | | | | Placebo/ | KHK4083 | | KHK4083 | | KHK4083 | | KHK4083 | | Placebo/ | |
| | KHK4083 | KHK4083 | KHK4083 | KHK4083 | KHK4083 | 150 mg | | 600 mg | | 300 mg | | 600 mg | | KHK4083 | |
| | 150 mg | 600 mg | 300 mg | 600 mg | 600 mg | Q4W | | Q4W | | Q2W | | Q2W | | 600 mg Q2W | |
| Visit | Q4W | Q4W | Q2W | Q2W | Q2W | N = 52 | | N = 52 | | N = 52 | | N = 54 | | N = 57 | |
| Week 24 | 40 | 40 | 41 | 42 | 30 | 25 | (48.1) | 28 | (53.8) | 34 | (65.4) | 29 | (53.7) | 11 | (19.3) |
| Week 26 | 38 | 38 | 39 | 43 | 26 | 25 | (48.1) | 29 | (55.8) | 34 | (65.4) | 33 | (61.1) | 17 | (29.8) |
| Week 28 | 36 | 38 | 38 | 40 | 26 | 26 | (50.0) | 26 | (50.0) | 33 | (63.5) | 32 | (59.3) | 17 | (29.8) |
| Week 30 | 34 | 37 | 38 | 40 | 25 | 24 | (46.2) | 26 | (50.0) | 32 | (61.5) | 32 | (59.3) | 18 | (31.6) |
| Week 32 | 33 | 38 | 37 | 40 | 23 | 23 | (44.2) | 29 | (55.8) | 31 | (59.6) | 35 | (64.8) | 19 | (33.3) |
| Week 34 | 33 | 38 | 35 | 37 | 23 | 23 | (44.2) | 27 | (51.9) | 30 | (57.7) | 31 | (57.4) | 19 | (33.3) |
| Week 36 | 34 | 37 | 36 | 37 | 25 | 27 | (51.9) | 30 | (57.7) | 33 | (63.5) | 31 | (57.4) | 20 | (35.1) |
| Week 40 | 26 | 34 | 30 | 30 | 22 | 23 | (44.2) | 28 | (53.8) | 27 | (51.9) | 26 | (48.1) | 17 | (29.0) |
| Week 44 | 24 | 34 | 29 | 30 | 19 | 21 | (40.4) | 27 | (51.9) | 29 | (55.6) | 25 | (46.3) | 17 | (29.8) |
| Week 48 | 22 | 32 | 28 | 26 | 17 | 19 | (36.5) | 30 | (57.7) | 28 | (53.8) | 24 | (44.4) | 15 | (26.3) |
| Week 52 | 18 | 33 | 23 | 24 | 17 | 16 | (30.8) | 30 | (57.7) | 22 | (42.3) | 22 | (40.7) | 14 | (24.6) |
| Week 56 | 19 | 28 | 18 | 20 | 16 | 15 | (28.8) | 24 | (46.2) | 16 | (30.8) | 17 | (31.5) | 14 | (24.6) |

### Proportion of Achievement for EASI-90 (Each Week):

TABLE 25
Variable: EASI-90

| | Number of Subjects with assessment | | | | | Number and (%) of Subjects Achieved | | | | | | | | | |
|---|---|---|---|---|---|---|---|---|---|---|---|---|---|---|---|
| | | | | | Placebo/ | KHK4083 | | KHK4083 | | KHK4083 | | KHK4083 | | Placebo/ | |
| | KHK4083 | KHK4083 | KHK4083 | KHK4083 | KHK4083 | 150 mg | | 600 mg | | 300 mg | | 600 mg | | KHK4083 | |
| | 150 mg | 600 mg | 300 mg | 600 mg | 600 mg | Q4W | | Q4W | | Q2W | | Q2W | | 600 mg Q2W | |
| Visit | Q4w | Q4W | Q2W | Q2W | Q2W | N = 52 | | N = 52 | | N = 52 | | N = 54 | | N = 57 | |
| Week 1 | 50 | 51 | 50 | 54 | 54 | 0 | (0.0) | 0 | (0.0) | 0 | (0.0) | 0 | (0.0) | 0 | (0.0) |
| Week 2 | 46 | 50 | 48 | 53 | 50 | 0 | (0.0) | 0 | (0.0) | 0 | (0.0) | 2 | (3.7) | 0 | (0.0) |
| Week 4 | 46 | 49 | 46 | 52 | 44 | 2 | (3.8) | 0 | (0.0) | 1 | (1.9) | 2 | (3.7) | 0 | (0.0) |
| Week 6 | 42 | 48 | 42 | 51 | 41 | 2 | (3.8) | 1 | (1.9) | 3 | (5.8) | 2 | (3.7) | 0 | (0.0) |
| Week 8 | 42 | 45 | 42 | 50 | 40 | 4 | (7.7) | 3 | (5.8) | 7 | (13.5) | 4 | (7.4) | 0 | (0.0) |
| Week 10 | 43 | 44 | 43 | 48 | 39 | 7 | (13.5) | 3 | (5.8) | 8 | (15.4) | 5 | (9.3) | 1 | (1.8) |
| Week 12 | 43 | 45 | 42 | 48 | 36 | 9 | (17.3) | 4 | (7.7) | 10 | (19.2) | 5 | (9.3) | 2 | (3.5) |
| Weak 14 | 42 | 42 | 42 | 48 | 34 | 6 | (11.5) | 6 | (11.5) | 16 | (30.8) | 8 | (14.8) | 2 | (3.5) |
| Weak 15 | 40 | 43 | 41 | 48 | 37 | 11 | (21.2) | 6 | (11.5) | 17 | (32.7) | 8 | (14.8) | 3 | (5.3) |
| Week 16 | 41 | 44 | 43 | 47 | 36 | 10 | (19.2) | 6 | (11.5) | 19 | (36.5) | 10 | (18.5) | 2 | (3.5) |
| Week 18 | 41 | 43 | 40 | 46 | 33 | 9 | (17.3) | 11 | (21.2) | 15 | (28.8) | 13 | (24.1) | 3 | (5.3) |
| Week 20 | 41 | 41 | 39 | 45 | 30 | 14 | (26.9) | 14 | (26.9) | 17 | (32.7) | 14 | (25.9) | 3 | (5.3) |
| Week 22 | 39 | 40 | 41 | 44 | 31 | 16 | (30.8) | 15 | (28.8) | 20 | (38.5) | 17 | (31.5) | 5 | (8.8) |

### Proportion of Achievement for EASI-90 (Each Week):

TABLE 26
Variable: EASI-90

| | Number of Subjects with assessment | | | | | Number and (%) of Subjects Achieved | | | | | | | | | |
|---|---|---|---|---|---|---|---|---|---|---|---|---|---|---|---|
| | | | | | Placebo/ | KHK4083 | | KHK4083 | | KHK4083 | | KHK4083 | | Placebo/ | |
| | KHK4083 | KHK4083 | KHK4083 | KHK4083 | KHK4083 | 150 mg | | 600 mg | | 300 mg | | 600 mg | | KHK4083 | |
| | 150 mg | 600 mg | 300 mg | 600 mg | 600 mg | Q4W | | Q4W | | Q2W | | Q2W | | 600 mg Q2W | |
| Visit | Q4W | Q4W | Q2W | Q2W | Q2W | N = 52 | | N = 52 | | N = 52 | | N = 54 | | N = 57 | |
| Week 24 | 40 | 40 | 41 | 42 | 30 | 14 | (26.9) | 14 | (26.9) | 26 | (50.0) | 16 | (29.6) | 6 | (10.5) |
| Week 26 | 38 | 38 | 39 | 43 | 26 | 14 | (26.9) | 16 | (30.8) | 26 | (50.0) | 20 | (37.0) | 6 | (10.5) |
| Week 28 | 36 | 38 | 38 | 40 | 26 | 15 | (28.8) | 14 | (26.9) | 25 | (48.1) | 17 | (31.5) | 5 | (8.8) |
| Week 30 | 34 | 37 | 38 | 40 | 25 | 14 | (26.9) | 18 | (34.6) | 25 | (48.1) | 24 | (44.4) | 8 | (14.0) |
| Week 32 | 33 | 38 | 37 | 40 | 23 | 16 | (30.8) | 17 | (32.7) | 27 | (51.9) | 25 | (46.3) | 11 | (19.3) |
| Week 34 | 33 | 38 | 35 | 37 | 23 | 19 | (36.5) | 15 | (28.8) | 27 | (51.9) | 24 | (44.4) | 11 | (19.3) |
| Week 36 | 34 | 37 | 36 | 37 | 25 | 18 | (34.6) | 19 | (36.5) | 28 | (53.8) | 21 | (38.9) | 13 | (22.8) |
| Week 40 | 26 | 34 | 30 | 30 | 22 | 15 | (28.8) | 19 | (36.5) | 23 | (44.2) | 18 | (33.3) | 13 | (22.8) |
| Week 44 | 24 | 34 | 29 | 30 | 19 | 14 | (26.9) | 24 | (46.2) | 24 | (46.2) | 20 | (37.0) | 12 | (21.1) |
| Week 48 | 22 | 32 | 28 | 26 | 17 | 14 | (26.9) | 21 | (40.4) | 22 | (42.3) | 18 | (33.3) | 11 | (19.3) |
| Week 52 | 18 | 33 | 23 | 24 | 17 | 12 | (23.1) | 24 | (46.2) | 17 | (32.7) | 19 | (35.2) | 11 | (19.3) |
| Week 56 | 19 | 28 | 18 | 20 | 16 | 13 | (25.0) | 21 | (40.4) | 13 | (25.0) | 15 | (27.8) | 8 | (14.0) |

### Proportion of Achievement for IGA0/1 (Each Week):

TABLE 27
Variable: Achievement of IGA Score of 0 or 1 with a reduction from Baseline >=2 Point

| | Number of Subjects with assessment | | | | | Number and (%) of Subjects Achieved | | | | | | | | | |
|---|---|---|---|---|---|---|---|---|---|---|---|---|---|---|---|
| | | | | | Placebo/ | KHK4083 | | KHK4083 | | KHK4083 | | KHK4083 | | Placebo/ | |
| | KHK4083 | KHK4083 | KHK4083 | KHK4083 | KHK4083 | 150 mg | | 600 mg | | 300 mg | | 600 mg | | KHK4083 | |
| | 150 mg | 600 mg | 300 mg | 600 mg | 600 mg | Q4W | | Q4W | | Q2W | | Q2W | | 600 mg Q2W | |
| Visit | Q4W | Q4W | Q2W | Q2W | Q2W | N = 52 | | N = 52 | | N = 52 | | N = 54 | | N = 57 | |
| Week 1 | 50 | 51 | 50 | 54 | 54 | 0 | (0.0) | 0 | (0.0) | 0 | (0.0) | 0 | (0. 0) | 0 | (0.0) |
| Week 2 | 46 | 50 | 18 | 53 | 50 | 0 | (0.0) | 0 | (0.0) | 1 | (1.9) | 1 | (1.9) | 0 | (0.0) |
| Week 4 | 46 | 49 | 46 | 52 | 44 | 1 | (1.9) | 1 | (1.9) | 0 | (0.0) | 3 | (5.6) | 1 | (1.8) |
| Week 6 | 42 | 48 | 42 | 51 | 41 | 2 | (3.8) | 2 | (3.8) | 1 | (1.9) | 2 | (3.7) | 0 | (0.0) |
| Week 8 | 42 | 45 | 42 | 50 | 40 | 5 | (9.6) | 2 | (3.8) | 2 | (3.8) | 2 | (3.7) | 0 | (0.0) |
| Week 10 | 43 | 44 | 43 | 48 | 39 | 6 | (11.5) | 4 | (7.7) | 6 | (11.5) | 2 | (3.7) | 1 | (1.8) |
| Week 12 | 43 | 45 | 42 | 48 | 36 | 9 | (17.3) | 6 | (11.5) | 12 | (23.1) | 4 | (7.4) | 1 | (1.8) |
| Week 14 | 42 | 42 | 42 | 4 8 | 34 | 8 | (15.4) | 5 | (9.6) | 11 | (21.2) | 4 | (7.4) | 2 | (3.5) |
| Week 15 | 40 | 43 | 41 | 48 | 37 | 10 | (19.2) | 7 | (13.5) | 9 | (17.3) | 8 | (14.8) | 2 | (3.5) |
| Week 16 | 41 | 44 | 43 | 47 | 36 | 10 | (19.2) | 8 | (15.4) | 16 | (30.8) | 10 | (18.5) | 1 | (1.8) |
| Week 18 | 41 | 43 | 40 | 46 | 33 | 10 | (19.2) | 8 | (15.4) | 16 | (30.8) | 12 | (22.2) | 1 | (1.8) |
| Week 20 | 41 | 41 | 39 | 45 | 30 | 13 | (25.0) | 9 | (17.3) | 15 | (28.8) | 14 | (25.9) | 3 | (5.3) |
| Week 22 | 39 | 40 | 41 | 44 | 31 | 13 | (25.0) | 12 | (23.1) | 18 | (34.6) | 14 | (25.9) | 3 | (5.3) |

### Proportion of Achievement for IGA0/1 (Each Week):

TABLE 28
Variable: Achievement of IGA Score of 0 or 1 with a reduction from Baseline of >=2 Point

| | Number of Subjects with assessment | | | | | Number and (%) of Subjects Achieved | | | | | | | | | |
|---|---|---|---|---|---|---|---|---|---|---|---|---|---|---|---|
| | | | | | Placebo/ | KHK4063 | | KHK4083 | | KHK4083 | | KHK4083 | | Placebo/ | |
| | KHK4083 | KHK4083 | KHK4083 | KHK4083 | KHK4083 | 150 mg | | 600 mg | | 300 mg | | 600 mg | | KHK4083 | |
| | 150 mg | 600 mg | 300 mg | 600 mg | 600 mg | Q4W | | Q4W | | Q2W | | Q2W | | 600 mg Q2W | |
| Visit | Q4W | Q4W | Q2W | Q2W | Q2W | N = 52 | | N = 52 | | N = 52 | | N = 54 | | N = 57 | |
| Week 24 | 40 | 40 | 41 | 42 | 30 | 14 | (26.9) | 10 | (19.2) | 20 | (38.5) | 12 | (22.2) | 3 | (5.3) |
| Week 26 | 38 | 38 | 39 | 43 | 26 | 13 | (25.0) | 14 | (26.9) | 25 | (48.1) | 18 | (33.3) | 4 | (7.0) |
| Week 28 | 36 | 38 | 38 | 40 | 26 | 11 | (21.2) | 12 | (23.1) | 23 | (44.2) | 14 | (25.9) | 3 | (5.3) |
| Week 30 | 34 | 37 | 38 | 40 | 25 | 13 | (25.0) | 12 | (23.1) | 22 | (42.3) | 17 | (31.5) | 4 | (7.0) |
| Week 32 | 33 | 38 | 37 | 40 | 23 | 14 | (26.9) | 13 | (25.0) | 24 | (46.2) | 23 | (42.6) | 5 | (8.8) |
| Week 34 | 33 | 38 | 35 | 37 | 23 | 16 | (30.8) | 11 | (21.2) | 24 | (46.2) | 20 | (37.0) | 7 | (12.3) |
| Week 36 | 34 | 37 | 36 | 38 | 25 | 18 | (34.6) | 14 | (26.9) | 27 | (51.9) | 19 | (35.2) | 8 | (14.0) |
| Week 40 | 26 | 34 | 30 | 30 | 22 | 10 | (19.2) | 17 | (32.7) | 19 | (36.5) | 13 | (24.1) | 7 | (12.3) |
| Week 44 | 24 | 34 | 29 | 30 | 19 | 12 | (23.1) | 16 | (30.8) | 25 | (48.1) | 16 | (29.6) | 11 | (19.3) |
| Week 48 | 22 | 32 | 28 | 26 | 17 | 8 | (15.4) | 21 | (40.4) | 20 | (38.5) | 18 | (33.3) | 9 | (15.8) |
| Weak 52 | 18 | 33 | 23 | 24 | 17 | 10 | (19.2) | 19 | (36.5) | 18 | (34.6) | 15 | (27.8) | 10 | (17.5) |
| Week 56 | 19 | 28 | 18 | 20 | 16 | 12 | (23.1) | 18 | (34.6) | 13 | (25.0) | 14 | (25.9) | 9 | (15.8) |

Percentage Changes (%) From Baseline in EASI Scores of Administration Groups in Each Week:

TABLE 29

| | | | KHK4083 | KHK4083 | KHK4083 | KHK4083 | Placebo/ |
|---|---|---|---|---|---|---|---|
| | | | 150 mg | 600 mg | 300 mg | 600 mg | KHK4083 |
| | | | Q4W | Q4W | Q2W | Q2W | 600 mg Q2W |
| Item | Visit | Statistics | N = 52 | N = 52 | N = 52 | N = 54 | N = 57 |
| Percent Changes | Week 1 | n | 50 | 51 | 50 | 54 | 54 |
| from Baseline | | Mean | −2.5 | −9.5 | −8.5 | −7.0 | −2.9 |
| | | SD | 22.3 | 20.6 | 34.5 | 28.0 | 29.4 |
| | | Min | −55 | −68 | −72 | −89 | −63 |
| | | Median | −1.9 | −2.2 | −1.0 | −5.6 | −3.6 |
| | | Max | 48 | 31 | 100 | 96 | 114 |
| | Week 2 | n | 46 | 50 | 48 | 53 | 50 |
| | | Mean | −6.7 | −14.3 | −18.4 | −15.6 | −2.6 |
| | | SD | 29.7 | 25.8 | 38.1 | 34.0 | 38.9 |
| | | Min | −71 | −75 | −88 | −100 | −72 |
| | | Median | −2.5 | −6.0 | −11.6 | −9.5 | −4.1 |
| | | Max | 71 | 31 | 128 | 79 | 154 |
| | Week 4 | n | 46 | 49 | 46 | 52 | 44 |
| | | Mean | −19.6 | −19.4 | −26.4 | −24.6 | −8.5 |
| | | SD | 39.8 | 28.3 | 46.2 | 34.4 | 26.8 |
| | | Min | −100 | −74 | −92 | −96 | −89 |
| | | Median | −14.9 | −14.8 | −27.6 | −18.3 | −6.6 |
| | | Max | 78 | 42 | 167 | 57 | 58 |

### Percentage Changes (%) From Baseline in EASI Scores of Administration Groups in Each Week:

TABLE 30

| | | | KHK4083 | KHK4083 | KHK4083 | KHK4083 | Placebo/ |
|---|---|---|---|---|---|---|---|
| | | | 150 mg | 600 mg | 300 mg | 600 mg | KHK4083 |
| | | | Q4W | Q4W | Q2W | Q2W | 600 mg Q2W |
| Iten | Visit | Statistics | N = 52 | N = 52 | N = 52 | N = 54 | N = 57 |
| Percent Changes | Week 6 | n | 42 | 48 | 42 | 51 | 41 |
| from Baseline | | Mean | −28.8 | −32.5 | −43.4 | −35.8 | −11.1 |
| | | SD | 37.6 | 34.6 | 42.0 | 35.1 | 35.6 |
| | | Min | −100 | −100 | −100 | −100 | −89 |
| | | Median | −26.3 | −29.7 | −52.9 | −32.5 | −8.7 |
| | | Max | 48 | 54 | 94 | 70 | 76 |
| | Week 8 | n | 42 | 45 | 42 | 50 | 40 |
| | | Mean | −40.3 | −45.5 | −52.2 | −42.1 | −19.2 |
| | | SD | 43.6 | 29.7 | 34.9 | 35.6 | 42.7 |
| | | Min | −100 | −100 | −97 | −100 | −83 |
| | | Median | −45.1 | −44.0 | −60.0 | −43.8 | −19.4 |
| | | Max | 118 | 29 | 37 | 50 | 150 |
| | Week 10 | n | 43 | 44 | 43 | 48 | 39 |
| | | Mean | =51.5 | −51.9 | −60.4 | −48.2 | −21.1 |
| | | SD | 35.4 | 33.8 | 33.0 | 34.5 | 38.4 |
| | | Min | −100 | −100 | −100 | −100 | −91 |
| | | Median | −58.6 | −55.8 | −69.8 | −50.0 | −20.8 |
| | | Max | 45 | 86 | 25 | 50 | 66 |

### Percentage Changes (%) From Baseline in EASI Scores of Administration Groups in Each Week:

TABLE 31

| | | | KHK4083 | KHK4083 | KHK4083 | KHK4083 | Placebo/ |
|---|---|---|---|---|---|---|---|
| | | | 150 mg | 600 mg | 300 mg | 600 mg | KHK4083 |
| | | | Q4W | Q4W | Q2W | Q2W | 600 mg Q2W |
| Iten | Visit | Statistics | N = 52 | N = 52 | N = 52 | N = 54 | N = 57 |
| Percent Changes | Week 12 | n | 43 | 45 | 42 | 48 | 36 |
| from Baseline | | Mean | −56.7 | −$8.4 | −68.3 | −$0.9 | −29.5 |
| | | SD | 33.8 | 26.5 | 28.1 | 31.9 | 39.7 |
| | | Min | −100 | −100 | −100 | −96 | −95 |
| | | Median | −60.0 | −63.6 | −76.4 | −55.5 | −32.4 |
| | | Max | 24 | 9 | 26 | 12 | 61 |
| | Week 14 | n | 42 | 42 | 42 | 48 | 34 |
| | | Mean | −62.5 | −62.1 | −71.1 | −61.5 | −32.1 |
| | | SD | 29.2 | 26.9 | 28.9 | 25.9 | 38.1 |
| | | Min | −100 | −100 | −100 | −100 | −95 |
| | | Median | −67.2 | −69.3 | −79.6 | −63.0 | −29.3 |
| | | Max | 7 | 12 | 36 | 0 | 30 |
| | Week 15 | n | 40 | 43 | 41 | 48 | 37 |
| | | Mean | −67.1 | −62.2 | −74.1 | −62.2 | −35.3 |
| | | SD | 29.4 | 28.5 | 24.9 | 27.3 | 41.4 |
| | | Min | −100 | −100 | −100 | −100 | −100 |
| | | Median | −72.4 | −66.7 | −81.0 | −67.3 | −41.2 |
| | | Max | 2 | 6 | −9 | 19 | 81 |

### Percentage Changes (%) From Baseline in EASI Scores of Administration Groups in Each Week:

TABLE 32

| | | | KHK4083 | KHK4083 | KHK4083 | KHK4083 | Placebo/ |
|---|---|---|---|---|---|---|---|
| | | | 150 mg | 600 mg | 300 mg | 600 mg | KHK4083 |
| | | | Q4W | Q4W | Q2W | Q2W | 600 mg Q2W |
| Item | Visit | Statistics | N = 52 | N = 52 | N = 52 | N = 54 | N = 57 |
| Percent Changes | Week 16 | n | 41 | 44 | 43 | 47 | 36 |
| from Baseline | | Mean | −67.0 | −63.2 | −77.2 | −63.6 | −37.4 |
| | | SD | 32.1 | 29.7 | 22.9 | 30.9 | 42.4 |
| | | Min | −100 | −100 | −100 | −100 | −100 |
| | | Median | −76.9 | −70.8 | −85.7 | −72.3 | −47.9 |
| | | Max | 18 | 6 | −20 | 52 | 81 |
| | Week 18 | n | 41 | 43 | 40 | 46 | 33 |
| | | Mean | −67.1 | −66.1 | −75.5 | −63.9 | −32.6 |
| | | SD | 30.8 | 27.5 | 23.3 | 52.3 | 46.6 |
| | | Min | −100 | −100 | −100 | −100 | −100 |
| | | Median | −78.6 | −70.0 | −85.2 | −75.1 | −48.0 |
| | | Max | 10 | −6 | −13 | 233 | 81 |
| | Week 20 | n | 41 | 41 | 39 | 45 | 30 |
| | | Mean | −69.7 | −70.6 | −79.0 | −74.4 | −37.9 |
| | | SD | 31.1 | 26.8 | 21.0 | 27.0 | 50.8 |
| | | Min | −100 | −100 | −100 | −100 | −100 |
| | | Median | −80.6 | −78.1 | −86.1 | −80.9 | −59.8 |
| | | Max | 24 | 0 | −12 | 11 | 95 |

### Percentage Changes (%) From Baseline in EASI Scores of Administration Groups in Each Week:

TABLE 33

| | | | KHK4083 | KHK4083 | KHK4083 | KHK4083 | Placebo/ |
|---|---|---|---|---|---|---|---|
| | | | 150 mg | 600 mg | 300 mg | 600 mg | KHK4083 |
| | | | Q4W | Q4W | Q2W | Q2W | 600 mg Q2W |
| Iten | Visit | Statistics | N = 52 | N = 52 | N = 52 | N = 54 | N = 57 |
| Percent Changes | Week 22 | n | 39 | 40 | 41 | 44 | 31 |
| from Baseline | | Mean | −74.3 | −75.6 | −80.8 | −79.4 | −49.3 |
| | | SD | 29.7 | 26.1 | 22.4 | 23.7 | 44.4 |
| | | Min | −100 | −100 | −100 | −100 | −100 |
| | | Median | −88.2 | −82.3 | −87.5 | −85.1 | −63.0 |
| | | Max | 24 | −5 | −12 | 33 | 95 |
| | Week 24 | n | 40 | 40 | 41 | 42 | 30 |
| | | Mean | −71.7 | −75.5 | −85.3 | −80.4 | −49.7 |
| | | SD | 32.0 | 25.9 | 22.0 | 22.5 | 46.1 |
| | | Min | −100 | −100 | −100 | −100 | −100 |
| | | Median | −81.7 | −83.8 | −92.0 | −85.4 | −60.0 |
| | | Max | 24 | −5 | 9 | 15 | 105 |
| | Week 26 | n | 38 | 38 | 39 | 43 | 26 |
| | | Mean | −75.3 | −78.2 | −86.3 | −81.0 | −70.1 |
| | | SD | 27.8 | 23.3 | 20.8 | 25.0 | 29.5 |
| | | Min | −100 | −100 | −100 | −100 | −100 |
| | | Median | −82.4 | −86.9 | −93.8 | −89.3 | −76.2 |
| | | Max | 24 | −23 | −18 | 15 | 20 |

Percentage Changes (%) From Baseline in EASI scores of Administration Groups in Each Week:

TABLE 34

| | | | KHK4083 | KHK4083 | KHK4083 | KHK4083 | Placebo/ |
|---|---|---|---|---|---|---|---|
| | | | 150 mg | 600 mg | 300 mg | 600 mg | KHK4083 |
| | | | Q4W | Q4W | Q2W | Q2W | 600 mg Q2W |
| Item | Visit | Statistics | N = 52 | N = 52 | N = 52 | N = 54 | N = 57 |
| Percent Changes | Week 28 | n | 36 | 38 | 38 | 40 | 26 |
| from Baseline | | Maan | −78.7 | −77.5 | 84.8 | −83.9 | −70.6 |
| | | SD | 26.1 | 22.1 | 26.3 | 19.6 | 27.5 |
| | | Min | −100 | −100 | −100 | −100 | −100 |
| | | Median | −85.6 | −85.5 | −94.3 | −89.0 | −82.6 |
| | | Max | 24 | −20 | 32 | −7 | 15 |
| | Week 30 | n | 34 | 37 | 38 | 40 | 25 |
| | | Mean | −80.8 | −81.1 | −87.3 | −86.5 | −74.5 |
| | | SD | 22.5 | 19.0 | 21.1 | 17.0 | 25.6 |
| | | Min | −100 | −100 | −100 | −100 | −100 |
| | | Median | −88.6 | −89.5 | −95.2 | −91.8 | −81.0 |
| | | Max | −12 | −31 | 11 | −14 | −6 |
| | Week 32 | n | 33 | 38 | 37 | 40 | 23 |
| | | Mean | −82.5 | −82.7 | −90.5 | −87.9 | −82.4 |
| | | SD | 23.9 | 17.6 | 13.4 | 14.4 | 17.4 |
| | | Min | −100 | −100 | −100 | −100 | −100 |
| | | Median | −89.3 | −88.6 | −96.4 | −91.9 | −88.0 |
| | | Max | 10 | −31 | −56 | −36 | −44 |

### Percentage Changes (%) From Baseline in EASI Scores of Administration Groups in Each Week:

TABLE 35

| | | | KHK4083 | KHK4083 | KHK4083 | KHK4083 | Placebo/ |
|---|---|---|---|---|---|---|---|
| | | | 150 mg | 600 mg | 300 mg | 600 mg | KHK4003 |
| | | | Q4W | Q4W | Q2W | Q2W | 600 mg Q2W |
| Item | Visit | Statistics | N = 52 | N = 52 | N = 52 | N = 54 | N = 57 |
| Percent Changes | Week 34 | n | 33 | 38 | 35 | 37 | 23 |
| from Baseline | | Mean | −83.6 | −80.5 | −91.3 | −87.1 | −81.0 |
| | | SD | 23.6 | 19.4 | 12.6 | 20.0 | 22.3 |
| | | Min | −100 | −100 | −100 | −100 | −100 |
| | | Median | −92.9 | −87.5 | −96.4 | −95.7 | −88.9 |
| | | Max | 16 | −23 | −52 | 4 | −18 |
| | Week 36 | n | 34 | 37 | 36 | 37 | 25 |
| | | Mean | −84.5 | −83.5 | −93.0 | −87.2 | −02.2 |
| | | SD | 23.1 | 20.1 | 10.0 | 14.3 | 23.2 |
| | | Min | −100 | −100 | −100 | −100 | −100 |
| | | Median | −94.0 | −90.0 | −96.5 | −90.9 | −90.4 |
| | | Max | 7 | −24 | −62 | −39 | 5 |
| | Week 40 | n | 26 | 34 | 30 | 30 | 22 |
| | | Mean | −88.0 | −86.9 | −91.7 | −88.4 | −82.3 |
| | | SD | 16.4 | 14.6 | 14.0 | 12.4 | 26.0 |
| | | Min | −100 | −100 | −100 | −100 | −100 |
| | | Median | −93.0 | −93.2 | −96.2 | −91.9 | −92.8 |
| | | Max | −36 | −52 | −33 | −54 | 0 |

### Percentage Changes (%) From Baseline in EASI Scores of Administration Groups in Each Week:

TABLE 36

| | | | KHK4083 | KHK4083 | KHK4083 | KHK4083 | Placebo/ |
|---|---|---|---|---|---|---|---|
| | | | 150 mg | 600 mg | 300 mg | 600 mg | KHK4083 |
| | | | Q4W | Q4W | Q2W | Q2W | 600 mg Q2W |
| Item | Visit | Statistics | N = 52 | N = 52 | N = 52 | N = 54 | N = 57 |
| Percent Changes | Week 44 | n | 24 | 34 | 29 | 30 | 19 |
| from Baseline | | Mean | −89.1 | −81.7 | −94.8 | −88.5 | −86.7 |
| | | SD | 18.1 | 38.8 | 6.0 | 15.0 | 23.3 |
| | | Min | −100 | −100 | −100 | −100 | −100 |
| | | Median | −93.2 | −94.9 | −96.7 | −95.0 | −94.4 |
| | | Max | −14 | 106 | −82 | −41 | 0 |
| | Week 48 | n | 22 | 32 | 28 | 26 | 17 |
| | | Mean | −84.5 | −90.8 | −94.3 | −91.6 | −85.0 |
| | | SD | 24.4 | 11.0 | 7.1 | 12.1 | 27.8 |
| | | Min | −100 | −100 | −100 | −100 | −100 |
| | | Median | −92.0 | −93.5 | −97.8 | −96.5 | −94.4 |
| | | Max | −11 | −56 | −78 | −57 | 12 |
| | Week 52 | n | 18 | 33 | 23 | 24 | 17 |
| | | Mean | −90.4 | −92.2 | −93.2 | −92.4 | −90.2 |
| | | SD | 14.1 | 12.4 | 9.4 | 10.8 | 12.8 |
| | | Min | −100 | −100 | −100 | −100 | −100 |
| | | Median | −94.2 | −97.4 | −96.7 | −94.3 | −100.0 |
| | | Max | −42 | −56 | −66 | −58 | −63 |

### Percentage Changes (%) From Baseline in EASI Scores of Administration Groups in Each Week:

TABLE 37

| | | | KHK4083 | KHK4083 | KHK4083 | KHK4083 | Placebo/ |
|---|---|---|---|---|---|---|---|
| | | | 150 mg | 600 mg | 300 mg | 600 mg | KHK4083 |
| | | | Q4W | Q4W | Q2W | Q2W | 600 mg Q2W |
| Item | Visit | Statistics | N = 52 | N = 52 | N = 52 | N = 54 | N = 57 |
| Percent Changes | Week 56 | n | 19 | 28 | 18 | 20 | 16 |
| from Baseline | | Mean | −87.4 | −88.3 | −90.3 | −90.4 | −88.3 |
| | | SD | 18.7 | 21.1 | 16.6 | 13.5 | 15.3 |
| | | Min | −100 | −100 | −100 | −100 | −100 |
| | | Median | −94.3 | −97.0 | −98.3 | −94.7 | −92.4 |
| | | Max | −32 | −35 | −36 | −57 | −50 |

### Time (Weeks) to Relapse Without KHK4083 Administration for Patients Achieving EASI-75 at Week 36:

TABLE 38
Achievement: EASI-75

| | | | | | | | | | Placebo/ | |
|---|---|---|---|---|---|---|---|---|---|---|
| | KHK4083 | | KHK4083 | | KHK4 083 | | KHK4 083 | | KHK4083 | |
| | 150 mg | | 600 mg | | 300 mg | | 600 mg | | 600 mg | |
| | Q4M | | CAN | | Q2H | | Q2W | | Q2W | |
| Statistics | N = 52 | | N = 52 | | N = 52 | | N = 54 | | N = 57 | |
| Subjects achieved at | 27 | | 30 | | 33 | | 31 | | 20 | |
| Week 36, n | | | | | | | | | | |
| Relapse, n (%) | 8 | (29.6) | 5 | (16.7) | 4 | (12.1) | 2 | (6.5) | 3 | (15.0) |
| Kaplan-Meier estimates (weeks) | | | | | | | | | | |
| 25th percentile (95% CI) | 16.14 | (7.43, 20.14) | — | (8.43, —) | — | (4.14, —) | — | (8.14, —) | — | (4.14, —) |
| Median (95% CI) | 20.14 | (16.14, —) | — | (—, —) | — | (—, —) | — | (—, —) | — | (—, —) |
| 75th percentile (95% CI) | — | (20.14, —) | — | (—, —) | — | (—, —) | — | (—, —) | — | (—, —) |
| Relapse probability (95% CI) at | | | | | | | | | | |
| 4 weeks from Week 36 | 3.8 | (0.6, 24.3) | 0.0 | (—, —) | 3.4 | (0.5, 22.1) | 0.0 | (—, —) | 0.0 | (—, —) |
| 8 weeks from Week 36 | 12.6 | (4.2, 34.2) | 6.9 | (1.8, 24.9) | 7.0 | (1.8, 25.3) | 0.0 | (—, —) | 5.6 | (0.8, 33.4) |
| 12 weeks from Week 36 | 17.2 | (6.8, 39.7) | 10.3 | (3.5, 28.7) | 7.0 | (1.8, 25.3) | 4.0 | (0.6, 25.2) | 12.3 | (3.2, 41.2) |
| 16 weeks from Week 36 | 22.4 | (9.9, 46.0) | 13.8 | (5.4, 32.7) | 7.0 | (1.8, 25.3) | 4.0 | (0.6, 25.2) | 12.3 | (3.2, 41.2) |
| 20 weeks from Week 36 | 27.5 | (13.3, 51.7) | 13.8 | (5.4, 32.7) | 11.7 | (3.9, 32.4) | 4.0 | (0.6, 25.2) | 19.0 | (6.5, 48.4) |

### Percentage Changes (%) From Baseline in EASI Scores of Administration Groups in Each Week Without Regard to Prohibited Concomitant Medications:

TABLE 39

| | | | KHK4083 | KHK4083 | KHK4083 | KHK4083 | Placebo/ |
|---|---|---|---|---|---|---|---|
| | | | 150 mg | 600 mg | 300 mg | 600 mg | KHK4083 |
| | | | Q4W | Q4W | Q2W | Q2W | 600 mg Q2W |
| Item | Visit | Statistics | N = 52 | N = 52 | N = 52 | N = 54 | N = 57 |
| Percent Changes | Week 1 | n | 50 | 51 | 50 | 54 | 54 |
| from Baseline | | Mean | −2.5 | −9.5 | −8.5 | −7.0 | −2.9 |
| | | SD | 22.3 | 20.6 | 34.5 | 28.8 | 29.4 |
| | | Min | −55 | −68 | −72 | −89 | −63 |
| | | Median | −1.9 | −2.2 | −1.0 | −5.6 | −3.6 |
| | | Max | 48 | 31 | 100 | 96 | 114 |
| | Week 2 | n | 47 | 50 | 50 | 53 | 50 |
| | | Mean | −7.4 | −14.3 | −16.7 | −15.6 | −2.6 |
| | | SD | 29.7 | 25.8 | 38.3 | 34.0 | 38.9 |
| | | Min | −71 | −75 | −88 | −100 | −72 |
| | | Median | −2.9 | −6.0 | −8.9 | −9.5 | −4.1 |
| | | Max | 71 | 31 | 128 | 79 | 154 |
| | Week 4 | n | 46 | 50 | 46 | 52 | 44 |
| | | Mean | −19.6 | −19.7 | −26.4 | −24.6 | −8.5 |
| | | SD | 39.8 | 28.1 | 46.2 | 34.4 | 26.8 |
| | | Min | −100 | −74 | −92 | −96 | −89 |
| | | Median | −14.9 | −15.2 | −27.6 | −18.3 | −6.6 |
| | | Max | 78 | 42 | 167 | 57 | 58 |

### Percentage Changes (%) From Baseline in EASI Scores of Administration Groups in Each Week Without Regard to Prohibited Concomitant Medications:

TABLE 40

| | | | KHK4083 | KHK4083 | KHK4083 | KHK4083 | Placebo/ |
|---|---|---|---|---|---|---|---|
| | | | 150 mg | 600 mg | 300 mg | 600 mg | KHK4083 |
| | | | Q4W | Q4W | Q2W | Q2W | 600 mg Q2W |
| Item | Visit | Statistics | N = 52 | N = 52 | N =52 | N = 54 | N = 57 |
| Percent Changes | Week 6 | n | 42 | 49 | 42 | 51 | 42 |
| from Baseline | | Mean | −28.8 | −32.6 | −43.4 | −35.8 | −12.2 |
| | | SD | 37.6 | 34.2 | 42.0 | 35.1 | 35.8 |
| | | Min | −100 | −100 | −100 | −100 | −89 |
| | | Median | −26.3 | −34.4 | −52.9 | −32.5 | −9.1 |
| | | Max | 48 | 54 | 94 | 70 | 76 |
| | Week 8 | n | 43 | 46 | 42 | 50 | 41 |
| | | Mean | −40.6 | −43.3 | −52.2 | −42.1 | −20.7 |
| | | SD | 43.1 | 33.0 | 34.9 | 35.6 | 43.3 |
| | | Min | −100 | −100 | −97 | −100 | −83 |
| | | Median | −46.0 | −43.9 | −60.0 | −43.8 | −20.0 |
| | | Max | 118 | 57 | 37 | 50 | 150 |
| | Week 10 | n | 43 | 44 | 43 | 48 | 39 |
| | | Mean | −51.5 | −51.9 | −60.4 | −48.2 | −21.1 |
| | | SD | 35.4 | 33.8 | 33.0 | 34.5 | 38.4 |
| | | Min | −100 | −100 | −100 | −100 | −91 |
| | | Median | −58.6 | −55.8 | −69.8 | −50.0 | −20.8 |
| | | Max | 45 | 86 | 25 | 50 | 66 |

### Percentage Changes (%) From Baseline in EASI Scores of Administration Groups in Each Week Without Regard to Prohibited Concomitant Medications:

TABLE 41

| | | | KHK4083 | KHK4083 | KHK4083 | KHK4083 | Placebo/ |
|---|---|---|---|---|---|---|---|
| | | | 150 mg | 600 mg | 300 mg | 600 mg | KHK4083 |
| | | | Q4W | Q4W | Q2W | Q2W | 600 mg Q2W |
| Item | Visit | Statistics | N = 52 | N = 52 | N = 52 | N = 54 | N = 57 |
| Percent Changes | Week 12 | n | 43 | 45 | 43 | 48 | 37 |
| from Baseline | | Mean | −56.7 | −58.4 | −68.2 | −50.9 | −31.0 |
| | | SD | 33.8 | 26.5 | 27.7 | 31.9 | 40.2 |
| | | Min | −100 | −100 | −100 | −96 | −95 |
| | | Median | −60.0 | −63.6 | −75.5 | −55.5 | −35.3 |
| | | Max | 24 | 9 | 26 | 12 | 61 |
| | Week 14 | n | 42 | 42 | 42 | 48 | 34 |
| | | Mean | −62.5 | −62.1 | −71.1 | −61.5 | −32.1 |
| | | SD | 29.2 | 26.9 | 28.9 | 25.9 | 38.1 |
| | | Min | −100 | −100 | −100 | −100 | −95 |
| | | Median | −67.2 | −69.3 | −79.6 | −63.0 | −29.3 |
| | | Max | 7 | 12 | 36 | 0 | 30 |
| | Week 15 | n | 40 | 43 | 41 | 48 | 37 |
| | | Mean | −67.1 | −62.2 | −74.1 | −62.2 | −35.3 |
| | | SD | 29.4 | 28.5 | 24.9 | 27.3 | 41.4 |
| | | Min | −100 | −100 | −100 | −100 | −100 |
| | | Median | −72.4 | −66.7 | −81.0 | −67.3 | −41.2 |
| | | Max | 2 | 6 | −9 | 19 | 81 |

### Percentage Changes (%) From Baseline in EASI Scores of Administration Groups in Each Week Without Regard to Prohibited Concomitant Medications:

TABLE 42

| | | | KHK4083 | KHK4083 | KHK4083 | KHK4083 | Placebo/ |
|---|---|---|---|---|---|---|---|
| | | | 150 mg | 600 mg | 300 mg | 600 mg | KHK4083 |
| | | | Q4W | Q4W | Q2W | Q2W | 600 mg Q2W |
| Item | Visit | Statistics | N = 52 | N = 52 | N = 52 | N = 54 | N = 57 |
| Percent Changes | Week 16 | n | 41 | 44 | 43 | 47 | 36 |
| from Baseline | | Mean | −67.0 | −63.2 | −77.2 | −63.6 | −37.4 |
| | | SD | 32.1 | 29.7 | 22.9 | 30.9 | 42.4 |
| | | Min | −100 | −100 | −100 | −100 | −100 |
| | | Median | −76.9 | −70.8 | −85.7 | −72.3 | −47.9 |
| | | Max | 18 | 6 | −20 | 52 | 81 |
| | Week 18 | n | 41 | 44 | 40 | 46 | 33 |
| | | Mean | −67.1 | −66.6 | −75.5 | −63.9 | −32.6 |
| | | SD | 30.8 | 27.5 | 23.3 | 52.3 | 46.6 |
| | | Min | −100 | −100 | −100 | −100 | −100 |
| | | Median | −78.6 | −74.1 | −85.2 | −75.1 | −48.0 |
| | | Max | 10 | −6 | −13 | 233 | 81 |
| | Week 20 | n | 41 | 41 | 39 | 45 | 30 |
| | | Mean | −69.7 | −70.6 | −79.0 | −74.4 | −37.9 |
| | | SD | 31.1 | 26.8 | 21.0 | 27.0 | 50.8 |
| | | Min | −100 | −100 | −100 | −100 | −100 |
| | | Median | −80.6 | −78.1 | −86.1 | −80.9 | −59.8 |
| | | Max | 24 | 0 | −12 | 11 | 95 |

Percentage Changes (%) From Baseline in EASI Scores of Administration Groups in Each Week Without Regard to Prohibited Concomitant Medications:

TABLE 43

| | | | KHK4083 | KHK4083 | KHK4083 | KHK4083 | Placebo/ |
|---|---|---|---|---|---|---|---|
| | | | 150 mg | 600 mg | 300 mg | 600 mg | KHK4083 |
| | | | Q4W | Q4W | Q2W | Q2W | 600 mg Q2W |
| Item | Visit | Statistics | N = 52 | N = 52 | N = 52 | N = 54 | N = 57 |
| Percent Changes | Week 22 | n | 39 | 40 | 41 | 44 | 31 |
| from Baseline | | Mean | −74.3 | −75.6 | −80.8 | −79.4 | −49.3 |
| | | SD | 29.7 | 26.1 | 22.4 | 23.7 | 44.4 |
| | | Min | −100 | −100 | −100 | −100 | −100 |
| | | Median | −88.2 | −82.3 | −87.5 | −85.1 | −63.0 |
| | | Max | 24 | −5 | −12 | 33 | 95 |
| | Week 24 | n | 40 | 40 | 41 | 43 | 30 |
| | | Mean | −71.7 | −75.5 | −85.3 | −79.9 | −49.7 |
| | | SD | 32.0 | 25.9 | 22.0 | 22.4 | 46.1 |
| | | Min | −100 | −100 | −100 | −100 | −100 |
| | | Median | −81.7 | −83.8 | −92.0 | −85.0 | −60.8 |
| | | Max | 24 | −5 | 9 | 15 | 105 |
| | Week 26 | n | 39 | 38 | 39 | 43 | 27 |
| | | Mean | −74.8 | −78.2 | −86.3 | −81.0 | −67.5 |
| | | SD | 27.6 | 23.3 | 20.8 | 25.0 | 31.9 |
| | | Min | −100 | −100 | −100 | −100 | −100 |
| | | Median | −82.4 | −86.9 | −93.8 | −89.3 | −76.0 |
| | | Max | 24 | −23 | −18 | 15 | 20 |

### Percentage Changes (%) From Baseline in EASI Scores of Administration Groups in Each Week Without Regard to Prohibited Concomitant Medications:

TABLE 44

| | | | KHK4083 | KHK4083 | KHK4083 | KHK4083 | Placebo/ |
|---|---|---|---|---|---|---|---|
| | | | 150 mg | 600 mg | 300 mg | 600 mg | KHK4083 |
| | | | Q4W | Q4W | Q2W | Q2W | 600 mg Q2W |
| Item | Visit | Statistics | N = 52 | N = 52 | N = 52 | N = 54 | N = 57 |
| Percent Changes | Week 28 | n | 36 | 38 | 38 | 40 | 26 |
| from Baseline | | Mean | −78.7 | −77.5 | −84.8 | −83.9 | −70.6 |
| | | SD | 26.1 | 22.1 | 26.3 | 19.6 | 27.5 |
| | | Min | −100 | −100 | −100 | −100 | −100 |
| | | Median | −85.6 | −85.5 | −94.3 | −89.0 | −82.6 |
| | | Max | 24 | −20 | 32 | −7 | 15 |
| | Week 30 | n | 34 | 37 | 38 | 40 | 25 |
| | | Mean | −80.8 | −81.1 | −87.3 | −86.5 | −74.5 |
| | | SD | 22.5 | 19.0 | 21.1 | 17.0 | 25.6 |
| | | Min | −100 | −100 | −100 | −100 | −100 |
| | | Median | −88.6 | −89.5 | −95.2 | −91.8 | −81.0 |
| | | Max | −12 | −31 | 11 | −14 | −6 |
| | Week 32 | n | 33 | 38 | 37 | 40 | 23 |
| | | Mean | −82.5 | −82.7 | −90.5 | −87.9 | −82.4 |
| | | SD | 23.9 | 17.6 | 13.4 | 14.4 | 17.4 |
| | | Min | −100 | −100 | −100 | −100 | −100 |
| | | Median | −89.3 | −88.6 | −96.4 | −91.9 | −88.0 |
| | | Max | 10 | −31 | −56 | −36 | −44 |

### Percentage Changes (%) From Baseline in EASI Scores of Administration Groups in Each Week Without Regard to Prohibited Concomitant Medications:

TABLE 45

| | | | KHK4083 | KHK4083 | KHK4083 | KHK4083 | Placebo/ |
|---|---|---|---|---|---|---|---|
| | | | 150 mg | 600 mg | 300 mg | 600 mg | KHK4083 |
| | | | Q4W | Q4W | Q2W | Q2W | 600 mg Q2W |
| Item | Visit | Statistics | N = 52 | N = 52 | N = 52 | N = 54 | N = 57 |
| Percent Changes | Week 34 | n | 33 | 38 | 35 | 37 | 23 |
| from Baseline | | Mean | −83.6 | −80.5 | −91.3 | −87.1 | −81.8 |
| | | SD | 23.6 | 19.4 | 12.6 | 20.0 | 22.3 |
| | | Min | −100 | −100 | −100 | −100 | −100 |
| | | Median | −92.9 | −87.5 | −96.4 | −95.7 | −88.9 |
| | | Max | 16 | −23 | −52 | 4 | −18 |
| | Week 36 | n | 34 | 37 | 36 | 37 | 25 |
| | | Mean | −84.5 | −83.5 | −93.0 | −87.2 | −82.2 |
| | | SD | 23.1 | 20.1 | 10.0 | 14.3 | 23.2 |
| | | Min | −100 | −100 | −100 | −100 | −100 |
| | | Median | −94.0 | −90.0 | −96.5 | −90.9 | −90.4 |
| | | Max | 7 | −24 | −62 | −39 | 5 |
| | Week 40 | n | 29 | 35 | 33 | 34 | 24 |
| | | Mean | −86.6 | −86.8 | −91.1 | −89.5 | −82.8 |
| | | SD | 17.6 | 14.5 | 13.7 | 12.1 | 25.1 |
| | | Min | −100 | −100 | −100 | −100 | −100 |
| | | Median | −92.9 | −92.6 | −95.7 | −92.9 | −92.8 |
| | | Max | −36 | −52 | −33 | −54 | 0 |

### Percentage Changes (%) From Baseline in EASI Scores of Administration Groups in Each Week Without Regard to Prohibited Concomitant Medications:

TABLE 46

| | | | KHK4083 | KHK4083 | KHK4083 | KHK4083 | Placebo/ |
|---|---|---|---|---|---|---|---|
| | | | 150 mg | 600 mg | 300 mg | 600 mg | KHK4083 |
| | | | Q4W | Q4W | Q2W | Q2W | 600 mg Q2W |
| Item | Visit | Statistics | N = 52 | N = 52 | N = 52 | N = 54 | N = 57 |
| Percent Changes | Week 44 | n | 30 | 35 | 36 | 36 | 23 |
| from Baseline | | Mean | −86.0 | −81.7 | −93.8 | −89.9 | −86.3 |
| | | SD | 22.6 | 38.2 | 7.2 | 14.8 | 21.9 |
| | | Min | −100 | 100 | −100 | −100 | −100 |
| | | Median | −93.2 | −94.6 | −96.3 | −95.6 | −94.4 |
| | | Max | −12 | 106 | −76 | −41 | 0 |
| | Week 48 | n | 30 | 36 | 35 | 37 | 23 |
| | | Mean | −81.0 | −84.4 | −93.7 | −91.6 | −87.0 |
| | | SD | 28.3 | 28.6 | 7.8 | 12.5 | 24.1 |
| | | Min | −100 | −100 | −100 | −100 | −100 |
| | | Median | −92.0 | −92.3 | −97.7 | −96.6 | −94.4 |
| | | Max | −11 | 66 | −78 | −53 | 12 |
| | Week 52 | n | 29 | 37 | 32 | 36 | 24 |
| | | Mean | −78.0 | −87.3 | −93.3 | −93.2 | −86.0 |
| | | SD | 30.2 | 22.9 | 9.0 | 9.7 | 22.7 |
| | | Min | −100 | −100 | −100 | −100 | −100 |
| | | Median | −94.1 | −96.0 | −97.2 | −95.2 | −90.9 |
| | | Max | 6 | 14 | −66 | −58 | 6 |

### Percentage Changes (%) From Baseline in EASI Scores of Administration Groups in in Each Week Without Regard to Prohibited Concomitant Medications:

TABLE 47

| | | | KHK4083 | KHK4083 | KHK4083 | KHK4083 | Placebo/ |
|---|---|---|---|---|---|---|---|
| | | | 150 mg | 600 mg | 300 mg | 600 mg | KHK4083 |
| | | | Q4W | Q4W | Q2W | Q2W | 600 mg Q2W |
| Item | Visit | Statistics | N = 52 | N = 52 | N = 52 | N = 54 | N = 57 |
| Percent Changes | Week 56 | n | 30 | 36 | 31 | 35 | 24 |
| from Baseline | | Mean | −77.1 | −83.4 | −88.6 | −91.2 | −81.8 |
| | | SD | 27.2 | 22.7 | 17.2 | 12.3 | 26.7 |
| | | Min | −100 | −100 | −100 | −100 | −100 |
| | | Median | −91.2 | −94.2 | −95.8 | −95.5 | −91.0 |
| | | Max | −12 | −24 | −36 | −57 | 12 |

### SEQUENCE LISTING FREE TEXT

SEQ ID NO 1: amino acid sequence of VH of KHK4083
SEQ ID NO 2: amino acid sequence of VL of KHK4083
SEQ ID NO 3: amino acid sequence of heavy chain constant region of KHK4083
SEQ ID NO 4: amino acid sequence of light chain constant region of KHK4083
SEQ ID NO 5: full length amino acid sequence of heavy chain of KHK4083
SEQ ID NO 6: full length amino acid sequence of light chain of KHK4083

1-10. (canceled)

11. A therapeutic method for an OX40-related immune- or allergy-related disease including subcutaneously administering an anti-OX40 antibody to a patient at a dose of 150 mg to 600 mg once in two weeks to four weeks continuously at the same dose.

12. The therapeutic method according to claim 11, wherein the anti-OX40 antibody is a monoclonal antibody containing a heavy chain variable region (also called VH) containing the amino acid sequence of SEQ ID NO: 1 and a light chain variable region (also called VL) containing the amino acid sequence of SEQ ID NO: 2.

13. The therapeutic method according to claim 11, wherein the administration is continued for at least 16 weeks, 20 weeks, 22 weeks, 24 weeks or 34 weeks after starting the administration.

14. The therapeutic method according to claim 11, wherein the OX40-related immune- or allergy-related disease is atopic dermatitis.

15. The therapeutic method according to claim 11, wherein the anti-OX40 antibody is subcutaneously administered once in two weeks, three weeks or four weeks.

16. The therapeutic method according to claim 11, wherein the dose is selected from 150 mg, 300 mg, 450 mg and 600 mg.

17. The therapeutic method according to claim 11, wherein the OX40-related immune- or allergy-related disease is moderate to severe atopic dermatitis.

18. The therapeutic method according to claim 11, wherein the OX40-related immune- or allergy-related disease is moderate to severe atopic dermatitis which is poorly controllable using a topical agent or moderate to severe atopic dermatitis for which a topical therapy is not medically recommended.

19. The therapeutic method according to claim 11 which is combined with a known topical agent such as a steroid.

20. The therapeutic method according to claim 11, wherein the anti-OX40 antibody is KHK4083.

21-40. (canceled)


SEQUENCE LISTING (6 sequences)

SEQ ID NO: 1 - protein, 117 aa, Homo sapiens
QITLKESGPTLVKPKQTLTLTCTFSGFSLSTSGMGVGWIRQPPGKALEWLAVIYWDDHQLYSPSLKSRLTITKDTSKNQVVLTMTNMDPVDTATYYCAHRRGAFQHWGQGTLVTVSS

SEQ ID NO: 2 - protein, 107 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYDSSLTFGGGTKVEIK

SEQ ID NO: 3 - protein, 330 aa, Homo sapiens [sequence omitted]

SEQ ID NO: 4 - protein, 107 aa, Homo sapiens [sequence omitted]

SEQ ID NO: 5 - protein, 447 aa, Homo sapiens
QITLKESGPTLVKPKQTLTLTCTFSGFSLSTSGMGVGWIRQPPGKALEWLAVIYWDDHQLYSPSLKSRLTITKDTSKNQVVLTMTNMDPVDTATYYCAHRRGAFQHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 214 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYDSSLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC